SEQUENCE LISTING (28 sequences)

SEQ ID NO: 1 - protein, 464 aa, Homo sapiens
MVRLAAELLLLLGLLLLTLHITVLRGSGAADGPDAAAGNASQAQLQNNLNVGSDTTSETSFSLSKEAPREHLDHQAAHQPFPRPRFRQETGHPSLQRDFPRSFLLDLPNFPDLSKADINGQNPNIQVTIEVVDGPDSEADKDQHPENKPSWSVPSPDWRAWWQRSLSLARANSGDQDYKYDSTSDDSNFLNPPRGWDHTAPGHRTFETKDQPEYDSTDGEGDWSLWSVCSVTCGNGNQKRTRSCGYACTATESRTCDRPNCPGIEDTFRTAATEVSLLAGSEEFNATKLFEVDTDSCERWMSCKSEFLKKYMHKVMNDLPSCPCSYPTEVAYSTADIFDRIKRKDFRWKDASGPKEKLEIYKPTARYCIRSMLSLESTTLAAQHCCYGDNMQLITRGKGAGTPNLISTEFSAELHYKVDVLPWIICKGDWSRYNEARPPNNGQKCTESPSDEDYIKQFQEAREY

SEQ ID NO: 2 - DNA, 1519 bp, Homo sapiens
taaagccgcgcgtctcaaaaggatggtgcgcctggcggccgagctgctgctgctgctggggctgctgctgctcacgctgcacatcaccgtgctgcgcggctcgggagccgccgacgggcccgacgcggccgcgggcaacgccagccaagcccagctgcagaataacctcaacgtgggaagtgacaccacatcagaaaccagcttttctctctccaaagaagcaccaagggagcatctggaccaccaggctgcacaccaacccttccccagaccgcgattccgacaagagacggggcacccttcattgcaaagagatttccccagatcctttctccttgatctaccaaactttccagatctttccaaagctgatatcaatgggcagaatccaaatatccaggtcaccatagaggtggtcgacggtcctgactctgaagcagataaagatcagcatccggagaataagcccagctggtcagtcccatcccccgactggcgggcctggtggcagaggtccctgtccttggccagggcaaacagcggggaccaggactacaagtacgacagtacctcagacgacagcaacttcctcaacccccccagggggtgggaccatacagccccaggccaccggacttttgaaaccaaagatcagccagaatatgattccacagatggcgagggtgactggagtctctggtctgtctgcagcgtcacctgcgggaacggcaaccagaaacggacccggtcttgtggctacgcgtgcactgcaacagaatcgaggacctgtgaccgtccaaactgcccaggaattgaagacacttttaggacagctgccaccgaagtgagtctgcttgcgggaagcgaggagtttaatgccaccaaactgtttgaagttgacacagacagctgtgagcgctggatgagctgcaaaagcgagttcttaaagaagtacatgcacaaggtgatgaatgacctgcccagctgcccctgctcctaccccactgaggtggcctacagcacggccgacatcttcgaccgcatcaagcgcaaggacttccgctggaaggacgccagcgggcccaaggagaagctggagatctacaagcccactgcccggtactgcatccgctccatgctgtccctggagagcaccacgctggcggcacagcactgctgctacggcgacaacatgcagctcatcaccaggggcaagggggcgggcacgcccaacctcatcagcaccgagttctccgcggagctccactacaaggtggacgtcctgccctggattatctgcaagggtgactggagcaggtataacgaggcccggcctcccaacaacggacagaagtgcacagagagcccctcggacgaggactacatcaagcagttccaagaggccagggaatattaaagagactgggatgaggtggaggacgctgcctctggttctggagcacacacgtgctgcactgacgtgccgactggcgccgagaccttcatagctgcggtcgtg

SEQ ID NO: 3 - protein, 164 aa, Homo sapiens
LFEVDTDSCERWMSCKSEFLKKYMHKVMNDLPSCPCSYPTEVAYSTADIFDRIKRKDFRWKDASGPKEKLEIYKPTARYCIRSMLSLESTTLAAQHCCYGDNMQLITRGKGAGTPNLISTEFSAELHYKVDVLPWIICKGDWSRYNEARPPNNGQKCTESPSDE

SEQ ID NO: 4 - DNA, 492 bp, Homo sapiens
ctgtttgaagttgacacagacagctgtgagcgctggatgagctgcaaaagcgagttcttaaagaagtacatgcacaaggtgatgaatgacctgcccagctgcccctgctcctaccccactgaggtggcctacagcacggccgacatcttcgaccgcatcaagcgcaaggacttccgctggaaggacgccagcgggcccaaggagaagctggagatctacaagcccactgcccggtactgcatccgctccatgctgtccctggagagcaccacgctggcggcacagcactgctgctacggcgacaacatgcagctcatcaccaggggcaagggggcgggcacgcccaacctcatcagcaccgagttctccgcggagctccactacaaggtggacgtcctgccctggattatctgcaagggtgactggagcaggtataacgaggcccggcctcccaacaacggacagaagtgcacagagagcccctcggacgag

SEQ ID NO: 5 - protein, 5 aa, Artificial Sequence
  RKD peptide
CRKDC

SEQ ID NO: 6 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 7 - DNA, 1965 bp, Homo sapiens
atgaagctctccctggtggccgcgatgctgctgctgctcagcgcggcgcgggccgaggaggaggacaagaaggaggacgtgggcacggtggtcggcatcgacctggggaccacctactcctgcgtcggcgtgttcaagaacggccgcgtggagatcatcgccaacgatcagggcaaccgcatcacgccgtcctatgtcgccttcactcctgaaggggaacgtctgattggcgatgccgccaagaaccagctcacctccaaccccgagaacacggtctttgacgccaagcggctcatcggccgcacgtggaatgacccgtctgtgcagcaggacatcaagttcttgccgttcaaggtggttgaaaagaaaactaaaccatacattcaagttgatattggaggtgggcaaacaaagacatttgctcctgaagaaatttctgccatggttctcactaaaatgaaagaaaccgctgaggcttatttgggaaagaaggttacccatgcagttgttactgtaccagcctattttaatgatgcccaacgccaagcaaccaaagacgctggaactattgctggcctaaatgttatgaggatcatcaacgagcctacggcagctgctattgcttatggcctggataagagggagggggagaagaacatcctggtgtttgacctgggtggcggaaccttcgatgtgtctcttctcaccattgacaatggtgtcttcgaagttgtggccactaatggagatactcatctgggtggagaagactttgaccagcgtgtcatggaacacttcatcaaactgtacaaaaagaagacgggcaaagatgtcaggaaagacaatagagctgtgcagaaactccggcgcgaggtagaaaaggccaaacgggccctgtcttctcagcatcaagcaagaattgaaattgagtccttctatgaaggagaagacttttctgagaccctgactcgggccaaatttgaagagctcaacatggatctgttccggtctactatgaagcccgtccagaaagtgttggaagattctgatttgaagaagtctgatattgatgaaattgttcttgttggtggctcgactcgaattccaaagattcagcaactggttaaagagttcttcaatggcaaggaaccatcccgtggcataaacccagatgaagctgtagcgtatggtgctgctgtccaggctggtgtgctctctggtgatcaagatacaggtgacctggtactgcttgatgtatgtccccttacacttggtattgaaactgtgggaggtgtcatgaccaaactgattccaaggaacacagtggtgcctaccaagaagtctcagatcttttctacagcttctgataatcaaccaactgttacaatcaaggtctatgaaggtgaaagacccctgacaaaagacaatcatcttctgggtacatttgatctgactggaattcctcctgctcctcgtggggtcccacagattgaagtcacctttgagatagatgtgaatggtattcttcgagtgacagctgaagacaagggtacagggaacaaaaataagatcacaatcaccaatgaccagaatcgcctgacacctgaagaaatcgaaaggatggttaatgatgctgagaagtttgctgaggaagacaaaaagctcaaggagcgcattgatactagaaatgagttggaaagctatgcctattctctaaagaatcagattggagataaagaaaagctgggaggtaaactttcctctgaagataaggagaccatggaaaaagctgtagaagaaaagattgaatggctggaaagccaccaagatgctgacattgaagacttcaaagctaagaagaaggaactggaagaaattgttcaaccaattatcagcaaactctatggaagtgcaggccctcccccaactggtgaagaggatacagcagaaaaagatgagttgtag

SEQ ID NO: 8 - protein, 1048 aa, Homo sapiens [sequence omitted]

SEQ ID NO: 9 - DNA, 3147 bp, Homo sapiens
atggcttttccgccgcggcgacggctgcgcctcggtccccgcggcctcccgcttcttctctcgggactcctgctacctctgtgccgtgccttcaacctagacgtggacagtcctgccgagtactctggccccgagggaagttacttcggcttcgccgtggatttcttcgtgcccagcgcgtcttcccggatgtttcttctcgtgggagctcccaaagcaaacaccacccagcctgggattgtggaaggagggcaggtcctcaaatgtgactggtcttctacccgccggtgccagccaattgaatttgatgcaacaggcaatagagattatgccaaggatgatccattggaatttaagtcccatcagtggtttggagcatctgtgaggtcgaaacaggataaaattttggcctgtgccccattgtaccattggagaactgagatgaaacaggagcgagagcctgttggaacatgctttcttcaagatggaacaaagactgttgagtatgctccatgtagatcacaagatattgatgctgatggacagggattttgtcaaggaggattcagcattgattttactaaagctgacagagtacttcttggtggtcctggtagcttttattggcaaggtcagcttatttcggatcaagtggcagaaatcgtatctaaatacgaccccaatgtttacagcatcaagtataataaccaattagcaactcggactgcacaagctatttttgatgacagctatttgggttattctgtggctgtcggagatttcaatggtgatggcatagatgactttgtttcaggagttccaagagcagcaaggactttgggaatggtttatatttatgatgggaagaacatgtcctccttatacaattttactggcgagcagatggctgcatatttcggattttctgtagctgccactgacattaatggagatgattatgcagatgtgtttattggagcacctctcttcatggatcgtggctctgatggcaaactccaagaggtggggcaggtctcagtgtctctacagagagcttcaggagacttccagacgacaaagctgaatggatttgaggtctttgcacggtttggcagtgccatagctcctttgggagatctggaccaggatggtttcaatgatattgcaattgctgctccatatgggggtgaagataaaaaaggaattgtttatatcttcaatggaagatcaacaggcttgaacgcagtcccatctcaaatccttgaagggcagtgggctgctcgaagcatgccaccaagctttggctattcaatgaaaggagccacagatatagacaaaaatggatatccagacttaattgtaggagcttttggtgtagatcgagctatcttatacagggccagaccagttatcactgtaaatgctggtcttgaagtgtaccctagcattttaaatcaagacaataaaacctgctcactgcctggaacagctctcaaagtttcctgttttaatgttaggttctgcttaaaggcagatggcaaaggagtacttcccaggaaacttaatttccaggtggaacttcttttggataaactcaagcaaaagggagcaattcgacgagcactgtttctctacagcaggtccccaagtcactccaagaacatgactatttcaagggggggactgatgcagtgtgaggaattgatagcgtatctgcgggatgaatctgaatttagagacaaactcactccaattactatttttatggaatatcggttggattatagaacagctgctgatacaacaggcttgcaacccattcttaaccagttcacgcctgctaacattagtcgacaggctcacattctacttgactgtggtgaagacaatgtctgtaaacccaagctggaagtttctgtagatagtgatcaaaagaagatctatattggggatgacaaccctctgacattgattgttaaggctcagaatcaaggagaaggtgcctacgaagctgagctcatcgtttctattccactgcaggctgatttcatcggggttgtccgaaacaatgaagccttagcaagactttcctgtgcatttaagacagaaaaccaaactcgccaggtggtatgtgaccttggaaacccaatgaaggctggaactcaactcttagctggtcttcgtttcagtgtgcaccagcagtcagagatggatacttctgtgaaatttgacttacaaatccaaagctcaaatctatttgacaaagtaagcccagttgtatctcacaaagttgatcttgctgttttagctgcagttgagataagaggagtctcgagtcctgatcatgtctttcttccgattccaaactgggagcacaaggagaaccctgagactgaagaagatgttgggccagttgttcagcacatctatgagctgagaaacaatggtccaagttcattcagcaaggcaatgctccatcttcagtggccttacaaatataataataacactctgttgtatatccttcattatgatattgatggaccaatgaactgcacttcagatatggagatcaaccctttgagaattaagatctcatctttgcaaacaactgaaaagaatgacacggttgccgggcaaggtgagcgggaccatctcatcactaagcgggatcttgccctcagtgaaggagatattcacactttgggttgtggagttgctcagtgcttgaagattgtctgccaagttgggagattagacagaggaaagagtgcaatcttgtacgtaaagtcattactgtggactgagacttttatgaataaagaaaatcagaatcattcctattctctgaagtcgtctgcttcatttaatgtcatagagtttccttataagaatcttccaattgaggatatcaccaactccacattggttaccactaatgtcacctggggcattcagccagcgcccatgcctgtgcctgtgtgggtgatcattttagcagttctagcaggattgttgctactggctgttttggtatttgtaatgtacaggatgggcttttttaaacgggtccggccacctcaagaagaacaagaaagggagcagcttcaacctcatgaaaatggtgaaggaaactcagaaacttaa

SEQ ID NO: 10 - protein, 799 aa, Homo sapiens
MPRAPAPLYACLLGLCALLPRLAGLNICTSGSATSCEECLLIHPKCAWCSKEDFGSPRSITSRCDLRANLVKNGCGGEIESPASSFHVLRSLPLSSKGSGSAGWDVIQMTPQEIAVNLRPGDKTTFQLQVRQVEDYPVDLYYLMDLSLSMKDDLDNIRSLGTKLAEEMRKLTSNFRLGFGSFVDKDISPFSYTAPRYQTNPCIGYKLFPNCVPSFGFRHLLPLTDRVDSFNEEVRKQRVSRNRDAPEGGFDAVLQAAVCKEKIGWRKDALHLLVFTTDDVPHIALDGKLGGLVQPHDGQCHLNEANEYTASNQMDYPSLALLGEKLAENNINLIFAVTKNHYMLYKNFTALIPGTTVEILDGDSKNIIQLIINAYNSIRSKVELSVWDQPEDLNLFFTATCQDGVSYPGQRKCEGLKIGDTASFEVSLEARSCPSRHTEHVFALRPVGFRDSLEVGVTYNCTCGCSVGLEPNSARCNGSGTYVCGLCECSPGYLGTRCECQDGENQSVYQNLCREAEGKPLCSGRGDCSCNQCSCFESEFGKIYGPFCECDNFSCARNKGVLCSGHGECHCGECKCHAGYIGDNCNCSTDISTCRGRDGQICSERGHCLCGQCQCTEPGAFGEMCEKCPTCPDACSTKRDCVECLLLHSGKPDNQTCHSLCRDEVITWVDTIVKDDQEAVLCFYKTAKDCVMMFTYVELPSGKSNLTVLREPECGNTPNAMTILLAVVGSILLVGLALLAIWKLLVTIHDRREFAKFQSERSRARYEMASNPLYRKPISTHTVDFTFNKFNKSYNGTVD

SEQ ID NO: 11 - DNA, 2400 bp, Homo sapiens
atgccgcgggccccggcgccgctgtacgcctgcctcctggggctctgcgcgctcctgccccggctcgcaggtctcaacatatgcactagtggaagtgccacctcatgtgaagaatgtctgctaatccacccaaaatgtgcctggtgctccaaagaggacttcggaagcccacggtccatcacctctcggtgtgatctgagggcaaaccttgtcaaaaatggctgtggaggtgagatagagagcccagccagcagcttccatgtcctgaggagcctgcccctcagcagcaagggttcgggctctgcaggctgggacgtcattcagatgacaccacaggagattgccgtgaacctccggcccggtgacaagaccaccttccagctacaggttcgccaggtggaggactatcctgtggacctgtactacctgatggacctctccctgtccatgaaggatgacttggacaatatccggagcctgggcaccaaactcgcggaggagatgaggaagctcaccagcaacttccggttgggatttgggtcttttgttgataaggacatctctcctttctcctacacggcaccgaggtaccagaccaatccgtgcattggttacaagttgtttccaaattgcgtcccctcctttgggttccgccatctgctgcctctcacagacagagtggacagcttcaatgaggaagttcggaaacagagggtgtcccggaaccgagatgcccctgaggggggctttgatgcagtactccaggcagccgtctgcaaggagaagattggctggcgaaaggatgcactgcatttgctggtgttcacaacagatgatgtgccccacatcgcattggatggaaaattgggaggcctggtgcagccacacgatggccagtgccacctgaacgaggccaacgagtacactgcatccaaccagatggactatccatcccttgccttgcttggagagaaattggcagagaacaacatcaacctcatctttgcagtgacaaaaaaccattatatgctgtacaagaattttacagccctgatacctggaacaacggtggagattttagatggagactccaaaaatattattcaactgattattaatgcatacaatagtatccggtctaaagtggagttgtcagtctgggatcagcctgaggatcttaatctcttctttactgctacctgccaagatggggtatcctatcctggtcagaggaagtgtgagggtctgaagattggggacacggcatcttttgaagtatcattggaggcccgaagctgtcccagcagacacacggagcatgtgtttgccctgcggccggtgggattccgggacagcctggaggtgggggtcacctacaactgcacgtgcggctgcagcgtggggctggaacccaacagtgccaggtgcaacgggagcgggacctatgtctgcggcctgtgtgagtgcagccccggctacctgggcaccaggtgcgagtgccaggatggggagaaccagagcgtgtaccagaacctgtgccgggaggcagagggcaagccactgtgcagcgggcgtggggactgcagctgcaaccagtgctcctgcttcgagagcgagttcggcaagatctatgggcctttctgtgagtgcgacaacttctcctgtgccaggaacaagggagtcctctgctcaggccatggcgagtgtcactgcggggaatgcaagtgccatgcaggttacatcggggacaactgtaactgctcgacagacatcagcacatgccggggcagagatggccagatctgcagcgagcgtgggcactgtctctgtgggcagtgccaatgcacggagccgggggcctttggggagatgtgtgagaagtgccccacctgcccggatgcatgcagcaccaagagagattgcgtcgagtgcctgctgctccactctgggaaacctgacaaccagacctgccacagcctatgcagggatgaggtgatcacatgggtggacaccatcgtgaaagatgaccaggaggctgtgctatgtttctacaaaaccgccaaggactgcgtcatgatgttcacctatgtggagctccccagtgggaagtccaacctgaccgtcctcagggagccagagtgtggaaacacccccaacgccatgaccatcctcctggctgtggtcggtagcatcctccttgttgggcttgcactcctggctatctggaagctgcttgtcaccatccacgaccggagggagtttgcaaagtttcagagcgagcgatccagggcccgctatgaaatggcttcaaatccattatacagaaagcctatctccacgcacactgtggacttcaccttcaacaagttcaacaaatcctacaatggcactgtggaatga

SEQ ID NO: 12 - DNA, 24 bp, Artificial Sequence
  Primer Forward ISM F59
taaagccgcgcgtctcaaaaggat

SEQ ID NO: 13 - DNA, 25 bp, Artificial Sequence
  Primer Reverse ISM R1577
cacgaccgcagctatgaaggtctcg

SEQ ID NO: 14 - DNA, 25 bp, Artificial Sequence
  Primer Forward GRP78
tctcgagatgaagctctccctggtg

SEQ ID NO: 15 - DNA, 30 bp, Artificial Sequence
  Primer reverse GRP78
ctggtaccgctacaactcatctttttctgc

SEQ ID NO: 16 - DNA, 31 bp, Artificial Sequence
  Primer forward ITGAV
cgaggcggccgccatggcttttccgccgcgg

SEQ ID NO: 17 - DNA, 28 bp, Artificial Sequence
  Primer reverse ITGAV
ctggtaccgttaagtttctgagtttcct

SEQ ID NO: 18 - DNA, 27 bp, Artificial Sequence
  Primer Forward ITGB5
tccaagcttatgccgcgggccccggcg

SEQ ID NO: 19 - DNA, 30 bp, Artificial Sequence
  Primer Reverse ITGB5
ccgggcggccgctcattccacagtgccatt

SEQ ID NO: 20 - protein, 435 aa, Homo sapiens
ADGPDAAAGNASQAQLQNNLNVGSDTTSETSFSLSKEAPREHLDHQAAHQPFPRPRFRQETGHPSLQRDFPRSFLLDLPNFPDLSKADINGQNPNIQVTIEVVDGPDSEADKDQHPENKPSWSVPSPDWRAWWQRSLSLARANSGDQDYKYDSTSDDSNFLNPPRGWDHTAPGHRTFETKDQPEYDSTDGEGDWSLWSVCSVTCGNGNQKRTRSCGYACTATESRTCDRPNCPGIEDTFRTAATEVSLLAGSEEFNATKLFEVDTDSCERWMSCKSEFLKKYMHKVMNDLPSCPCSYPTEVAYSTADIFDRIKRKDFRWKDASGPKEKLEIYKPTARYCIRSMLSLESTTLAAQHCCYGDNMQLITRGKGAGTPNLISTEFSAELHYKVDVLPWIICKGDWSRYNEARPPNNGQKCTESPSDEDYIKQFQEAREY

SEQ ID NO: 21 - protein, 5 aa, artificial sequence
  Peptide
CRAAC

SEQ ID NO: 22 - protein, 7 aa, artificial sequence
  Peptide
CKRKDFC

SEQ ID NO: 23 - protein, 14 aa, artificial sequence
  Peptide
CDRIKRKDFRWKDC

SEQ ID NO: 24 - protein, 5 aa, artificial sequence
  Peptide
CAKDC

SEQ ID NO: 25 - protein, 5 aa, artificial sequence
  Peptide
CRADC

SEQ ID NO: 26 - protein, 5 aa, artificial sequence
  Peptide
CRKAC

SEQ ID NO: 27 - protein, 5 aa, artificial sequence
  Cilengitide
RGDFV

SEQ ID NO: 28 - protein, 5 aa, artificial sequence
  Peptide
RKLYD

(19) United States
(12) Patent Application Publication
(10) Pub. No.: US 2016/0002297 A1
(43) Pub. Date: Jan. 7, 2016

(54) THERAPEUTIC AGENTS

(71) Applicant: NATIONAL UNIVERSITY OF SINGAPORE, Singapore (SG)

(72) Inventors: Ruowen Ge, Singapore (SG); Mo Chen, Singapore (SG)

(73) Assignee: National University of Singapore, Singapore (SG)

(21) Appl. No.: 14/769,033

(22) PCT Filed: Feb. 18, 2014
(86) PCT No.: PCT/SG2014/000062

§ 371 Date: Aug. 19, 2015

Related U.S. Application Data
(60) Provisional application No. 61/851,412, filed on Mar. 7, 2013.

(30) Foreign Application Priority Data
Feb. 19, 2013 (GB) 1302837.8

(51) Int. Cl. C07K 7/64; A61K 51/08; A61K 49/00; A61K 38/12; A61K 45/06

(52) U.S. Cl. CPC C07K 7/64; A61K 38/12; A61K 45/06; A61K 49/0056; A61K 51/08

(57) ABSTRACT

This disclosure relates to therapeutic agents comprising polypeptides and peptides that include the amino acid sequence motif arginine-lysine-aspartic acid [RKD] and their use in the treatment of conditions associated with abnormal angiogenesis.

### FIELD OF THE INVENTION

This disclosure relates to therapeutic agents comprising polypeptides and peptides that include the amino acid sequence motif arginine-lysine-aspartic acid [RKD] and their use in the treatment of conditions associated with abnormal angiogenesis, such as cancer and including methods of diagnosis or prognosis that can be optionally combined with a method of treatment.

### BACKGROUND TO THE INVENTION

Angiogenesis, the formation of new blood vessels, is critical in embryogenesis, wound healing and reproduction but also plays a major role in tumour progression and metastasis. Metastasis describes the spread of a cancer from one organ or part to another organ. Once metastasis has occurred, the chances of survival decrease drastically and therefore effective angiogenesis inhibitors that specifically inhibit cancer progression are urgently sought after. Angiogenesis inhibitors such as Avastin®, which targets all isoforms of the human vascular endothelial growth factor A (VEGF) are being used in antibody therapy to prevent tumour growth and metastasis. However, these antibodies are typically very expensive to produce and can have undesirable side effects such as deferred wound healing, clots in the arteries, hypertension and protein in the urine. Abnormal angiogenesis also occurs in other conditions such as diabetes mellitus, rheumatoid arthritis, macular degeneration and psoriasis.

Other common treatment methods for cancer include radiotherapy or the use of chemotherapeutic agents. Traditional chemotherapeutic agents are toxins which act by killing cells that divide uncontrollably, one of the main properties of most cancer cells. However, non-cancerous dividing cells such as cells in the bone marrow, digestive tract, and hair follicles are also targeted by this treatment. This results in severe side-effects, for example a high temperature, shivering, breathing difficulties, flu-like symptoms, such as muscle aches and pain, bleeding gums or nose, severe vomiting, diarrhoea, immunosuppression and alopecia. Chemotherapy has its limitations and is less effective once the cancer has spread to other organs or parts of the body.

Isthmin (ISM) a 60 kDa secreted-angiogenesis inhibitor was found to suppress tumour growth in mice and disrupts vessel patterning in zebrafish embryos. ISM binds selectively, with low affinity to αvβ5 integrin on the surface of endothelial cells (ECs) through a ‘RKD’ motif, and induces EC apoptosis through integrin mediated death (IMD) by direct recruitment and activation of caspase-8. Immobilized ISM, however, loses its anti-angiogenic function and instead promotes EC adhesion, survival and migration through αvβ5 integrin by activating focal adhesion kinase (FAK). ISM has therefore unexpectedly both pro-survival and death-promoting effects on ECs depending on its physical state. WO2009/113965 discloses the use of ISM derivatives containing the AMOP domain for the use in treating conditions associated with abnormal angiogenesis.

The glucose-regulated protein GRP78, also referred to as BiP (immunoglobulin heavy-chain binding protein), is a cellular protein induced by glucose starvation. Residing primarily in the ER, GRP78 belongs to the HSP70 protein family. This disclosure relates to the discovery that GRP78 is a high-affinity receptor for ISM, mediating the internalization of ISM through clathrin-dependent endocytosis and navigating ISM to mitochondria. Inside the mitochondria, ISM blocks ATP transport from mitochondria to cytosol by interacting and inhibiting ADP/ATP carriers (AACs). This GRP78-mediated internalization is crucial for the pro-apoptotic function of ISM. Thus, soluble ISM induces apoptosis through cell-surface GRP78 mediated mitochondrial-targeting and induction of mitochondrial dysfunction.

With increasing understanding of the molecular signatures of various cancers, a biomarker which can indicate cancer aggressiveness and potential chemoresistance in each individual cancer patient is critical for guiding effective cancer treatment. The age of theranostics has arrived, designing individualized treatment based on the diagnostics and prognostics signature of a cancer patient. Hence, reliable biomarkers for cancer behavior as well as diagnostic agents to analyze these biomarkers in clinics are urgently needed.

Many human cancers at advanced stage/metastatic stage overexpress the glucose-regulated protein 78 kDa (GRP78), an ER resident chaperone protein involved in facilitating protein folding. Most importantly, when GRP78 is overexpressed, a fraction of GRP78 in these cancers is translocated onto the cell-surface and serves as a receptor for cell signalling. Cancers which overexpress GRP78 include breast cancer, prostate cancer, colon cancer, stomach cancer, lung cancer and liver cancer and others. In contrast, cells in normal tissues and organs do not harbour GRP78 on their cell-surface. Furthermore, emerging evidence suggests that cell-surface GRP78 is preferentially present on cancer stem cells, thus presenting a potential opportunity to target cancer stem cells. Cancer stem cells are believed to be chemoresistant and are most difficult to eliminate.

Cancer is an immensely heterogeneous disease and as a result, existing treatments are often effective for only limited patient subpopulations and at certain stages of disease. As a prognostic biomarker, GRP78 may be indicative of aggressive growth, high metastatic potential and increased resistance to chemotherapy. This may in turn translate to a higher risk of disease recurrence or progression which may necessitate modification of treatment to improve patient outcome. Thus, an imaging probe that can determine the level of cell-surface GRP78 in an individual cancer patient through non-invasive method such as PET scan may potentially be very useful for cancer treatment. Currently, no such imaging agent is available. A high-affinity ligand of cell-surface GRP78 thus has great potential to be a cancer companion diagnostic/prognostic/predictive agent to determine cancer aggressiveness and chemoresistance.

Radiolabelled ISM or its derivatives can function as a PET imaging probe by binding to cell-surface GRP78. Cell-surface GRP78 level can serve as cancer companion diagnostic/prognostic/predictive marker in patients with multiple types of cancer including but not limited to cancers of liver, lung, prostate, stomach, breast and colon.

This disclosure demonstrates that the RKD motif alone is sufficient to inhibit in vitro angiogenesis and induces endothelial cell apoptosis. Furthermore, cyclized CRKDC peptide potently suppresses subcutaneous mouse melanoma and breast cancer growth by 75% when delivered systematically through an intravenous injection. Due to its small size the peptide is highly stable in vitro and in vivo, can easily be synthesized in large quantity and has therefore high economic value. The different binding affinities of ISM for GRP78 and αvβ5 integrin are also of importance as different dosage regimens can alter the target specificity making ISM and derived peptides suitable as an anti-angiogenic and anti-cancer drug. Moreover, this disclosure relates to polypeptides and peptides comprising the RKD motif that recognize a biomarker GRP78 that can be used in the diagnosis or prognosis of cancer.

### STATEMENT OF THE INVENTION

According to an aspect of the invention there is provided a therapeutic agent comprising a polypeptide or peptide comprising the amino acid motif arginine-lysine-aspartic acid [RKD] for use in the treatment of excessive or abnormal angiogenesis conditions wherein cells associated with said condition express or overexpresses the glucose-regulated protein GRP78.

In a preferred embodiment of the invention said agent comprises a polypeptide comprising an amino acid sequence as set forth in SEQ ID NO: 1 or 20, or an amino acid sequence variant wherein said variant is modified by addition, deletion or substitution of one or more amino acid residues and wherein said polypeptide has retained or enhanced binding to GRP78.

In a preferred embodiment of the invention said agent comprises a polypeptide comprising the amino acid sequences 289-452 of SEQ ID NO: 1.

In a preferred embodiment of the invention said agent comprises a polypeptide consisting essentially of the amino acid sequence set forth in SEQ ID NO: 3.

A variant, i.e. a fragment polypeptide and reference polypeptide may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like character. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan. Most highly preferred are variants which retain the same biological function and activity as the reference polypeptide from which it varies.

A functionally equivalent polypeptide of SEQ ID NO: 1 or 3 or 20 is a variant in which one or more amino acid residues are substituted with conserved or non-conserved amino acid residues, or a variant in which one or more amino acid residues includes a substituent group. Conservative substitutions are the replacements, one for another, among the aliphatic amino acids Ala, Val, Leu and Ile; interchange of the hydroxyl residues Ser and Thr; exchange of the acidic residues Asp and Glu; substitution between amide residues Asn and Gln; exchange of the basic residues Lys and Arg; and replacements among aromatic residues Phe and Tyr.

In addition, the invention features polypeptide sequences having at least 75% identity with the polypeptide sequences illustrated in SEQ ID NO: 1 or 3 or 20, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated in SEQ ID NO: 1 or 3 or 20.

In an alternative preferred embodiment of the invention said agent is a peptide comprising at least the amino acid motif RKD.

In a preferred embodiment of the invention said agent comprises a peptide between 3 and 163 amino acids.

In a preferred embodiment of the invention said agent comprises a peptide that is 5, 10, 20, 25, 30, 40, 50, 60, 70, 80, 90, 100, 110, 120, 130, 140, 150 or 160 amino acids in length.

In a preferred embodiment said agent is a cyclic peptide.

Cyclisation is known in the art, (see Scott et al Chem Biol (2001), 8:801-815; Gellerman et al J. Peptide Res (2001), 57: 277-291; Dutta et al J. Peptide Res (2000), 8: 398-412; Ngoka and Gross J Amer Soc Mass Spec (1999), 10:360-363.

According to a further aspect of the invention there is provided an agent comprising a cyclic peptide comprising the amino acid motif arginine-lysine-aspartic acid [RKD] for use as a medicament.

In a preferred embodiment of the invention said agent comprises a peptide comprising the amino acid motif cysteine-arginine-lysine-aspartic acid-cysteine [SEQ ID NO: 5].

In a preferred embodiment of the invention said peptide consists of the amino acid motif cysteine-arginine-lysine-aspartic acid-cysteine.

In a preferred embodiment of the invention said agent includes more than one amino acid motif comprising the amino acid sequence RKD.

In a preferred embodiment of the invention said agent comprises a polypeptide, peptide or cyclic peptide that is pegylated.

In an alternative preferred embodiment of the invention said agent comprises a peptide comprising one or more non-natural amino acid residues.

According to a further aspect of the invention there is provided an agent comprising a fusion protein comprising a therapeutic polypeptide or peptide according to the invention translationally fused to an Fc portion of immunoglobulin.

According to a further aspect of the invention there is provided a pharmaceutical composition comprising an agent according to the invention including a pharmaceutically acceptable excipient and/or carrier.

When administered, the compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents, such as chemotherapeutic agents.

The therapeutics of the invention can be administered by any conventional route, including injection or by gradual infusion over time. The administration may, for example, be oral, intravenous, intraperitoneal, intramuscular, intracavity, subcutaneous, transdermal or an aerosol [e.g. for delivery to the lungs] and sublingual. Techniques for preparing aerosol delivery systems containing therapeutic polypeptides or peptides according to the invention are well known to those of skill in the art. Generally, such systems should utilize components which will not significantly impair the biological properties of the polypeptides or peptides. Those of skill in the art can readily determine the various parameters and conditions for producing aerosols without resort to undue experimentation.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of therapeutic polypeptides or peptides according to the invention for producing the desired response in a unit of weight or volume suitable for administration to a patient. The response can, for example, be measured by determining the signal transduction enhanced or inhibited by the composition via a reporter system, by measuring downstream effects such as gene expression, or by measuring the physiological effects of the therapeutic polypeptide or peptide composition, such as regression of a tumor, decrease of disease symptoms, modulation of apoptosis, etc. Other assays will be known to one of ordinary skill in the art and can be employed for measuring the level of the response.

The doses of the therapeutic polypeptide or peptide administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of therapeutic polypeptide or peptide are formulated and administered in doses between 1 ng and 1 mg, and preferably between 10 ng and 100 μg, according to any standard procedure in the art. Other protocols for the administration of polypeptide or peptide compositions will be known to one of ordinary skill in the art, in which the dose amount, schedule of injections, sites of injections, mode of administration (e.g., intra-tumoral) and the like vary from the foregoing. Administration of compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of polypeptides or peptides which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

In a preferred embodiment of the invention said composition comprises an additional, different therapeutic agent.

Preferably, said additional therapeutic agent is an anti-cancer agent, for example a chemotherapeutic agent or anti-angiogenic agent which is targeted to cancer cells or endothelial cells.

In a preferred embodiment of the invention said additional agent is cross-linked or associated with the therapeutic polypeptide or peptide according to the invention.

According to a further aspect of the invention there is provided an agent according to the invention for use in the treatment of conditions that would benefit from the inhibition of abnormal angiogenesis.

In a preferred embodiment of the invention said condition is cancer; preferably metastatic cancer.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In a preferred embodiment of the invention said cancer comprises cells that express or over expresses glucose regulated protein 78 (GRP78).

In a preferred embodiment of the invention said cancer is selected from the group consisting of: liver, prostate, skin [e.g. melanoma], breast and colon cancer.

In an alternative preferred embodiment of the invention said condition is diabetes mellitus, for example diabetic retinopathy or nephropathy.

In a further preferred embodiment of the invention said condition is rheumatoid arthritis.

In a preferred embodiment of the invention said condition is psoriasis.

In a further preferred embodiment of the invention said condition is an eye condition selected from the group: age related macular degeneration, neovascular glaucoma, corneal neovascularization [trachoma] and pterygium

According to a further aspect of the invention there is provided a method to diagnose and treat a subject that has or has a predisposition to a disease associated with excessive or abnormal angiogenesis comprising the steps of:
i) providing an isolated biological sample to be tested and preparing cDNA;
ii) forming a preparation comprising said cDNA and an oligonucleotide primer pair adapted to anneal to a nucleic acid molecule comprising SEQ ID NO: 7, 9 or 11; a thermostable DNA polymerase, deoxynucleotide triphosphates and co-factors;
iii) providing polymerase chain reaction conditions sufficient to amplify all or part of said nucleic acid molecule;
iv) analysing the amplified products of said polymerase chain reaction for the presence of a nucleic acid molecule comprising a nucleotide sequence derived from SEQ ID NO: 7, 9 or 11; and optionally
v) comparing the amplified product with a normal matched control.

In a preferred method of the invention said method is a real time PCR method for the detection and quantification of a nucleic acid encoding all or part of the nucleotide sequence set forth in SEQ ID NO: 7, 9 or 11.

In a preferred method of the invention said oligonucleotide primer pairs are selected from the group consisting of:

[SEQ ID NO: 14]
5′ TCTCGAGATGAAGCTCTCCCTGGTG 3′
[SEQ ID NO: 15]
5′ CTGGTACCGCTACAACTCATCTTTTTCTGC 3′
[SEQ ID NO: 16]
5′ CGAGGCGGCCGCCATGGCTTTTCCGCCGCGG 3′
[SEQ ID NO: 17]
5′ CTGGTACCGTTAAGTTTCTGAGTTTCCT 3′
[SEQ ID NO: 18]
5′ TCCAAGCTTATGCCGCGGGCCCCGGCG 3′
[SEQ ID NO: 19]
5′ CCGGGCGGCCGCTCATTCCACAGTGCCATT 3′.

According to a further aspect of the invention there is provided a method to diagnose and treat a subject that has or has a predisposition to a disease associated with excessive or abnormal angiogenesis comprising the steps of:
i) providing an isolated biological sample to be tested;
ii) forming a preparation comprising said sample and an antibody, or antibodies, that specifically bind a polypeptide in said sample as represented by the amino acid sequences presented in SEQ ID NO: 6, 8 or 10 to form an antibody/polypeptide complex;
iii) detecting the complex; and
iv) comparing the expression of said polypeptide with a normal matched control.

In a preferred method of the invention said biological sample is selected from the group consisting of: blood, blood plasma or serum, lymph fluid, saliva, sputum, lavage, urine, semen or a tissue biopsy or including prostate, ovary, bladder, colon, lung, bone, skin and breast.

In a preferred method of the invention said method further comprises designing a treatment regimen for the prevention or treatment of a condition that would benefit from inhibition of angiogenesis as determined by the result of said diagnostic method.

In a preferred method of the invention said treatment regimen comprises administration of an agent or pharmaceutical composition according to the invention.

According to a further aspect of the invention there is provided an agent according to the invention crosslinked or associated with one or more imaging agents.

In a further preferred embodiment said imaging reagents are selected from the group: green fluorescent protein, yellow fluorescent protein, red fluorescent protein.

An “imaging agent” is an agent capable of detection, for example by spectrophotometry, flow cytometry, or microscopy. For example, a label can be attached to the polypeptide or peptide, thereby permitting detection of the polypeptide or peptide in viva Examples of imaging agents include, but are not limited to, radioactive isotopes, enzyme substrates, co-factors, ligands, chemiluminescent agents, fluorophores, haptens, enzymes, and combinations thereof. Methods for labelling and guidance in the choice of labels appropriate for various purposes are discussed for example in Sambrook et al. (Molecular Cloning: A Laboratory Manual, Cold Spring Harbour, New York, 1989) and Ausubel et al. (In Current Protocols in Molecular Biology, John Wiley & Sons, New York, 1998). Fluorophores are imaging agents commonly used in the art. A fluorophore is a chemical compound, which when excited by exposure to a particular stimulus, such as a defined wavelength of light, emits light (fluoresces), for example at a different wavelength (such as a longer wavelength of light). Fluorophores are part of the larger class of luminescent compounds. Luminescent compounds include chemiluminescent molecules, which do not require a particular wavelength of light to luminesce, but rather use a chemical source of energy. Therefore, the use of chemiluminescent molecules eliminates the need for an external source of electromagnetic radiation, such as a laser.

In a preferred embodiment of the invention said imaging agent comprises a luminescent molecule.

In a preferred embodiment of the invention said imaging agent comprises a fluorescence molecule.

In a preferred embodiment of the invention said fluorescence molecule is a fluorescent dye.

In a preferred embodiment said fluorescence molecule is a fluorescent protein.

In an alternative preferred embodiment of the invention said imaging agent comprises a radioisotope.

According to a further aspect of the invention there is provided a method to image a tumour comprising:
i) administration of an imaging agent according to the invention to a subject; and
ii) detecting the imaging agent bound to GPR78 expressed by a tumour cell, tumor endothelial cell and/or cancer stem cell.

In a preferred method of the invention said method is single-photon emission computed tomography.

In an alternative preferred method of the invention said method is positron emission tomography.

In a preferred method of the invention said method is fluorescence microscopy. Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

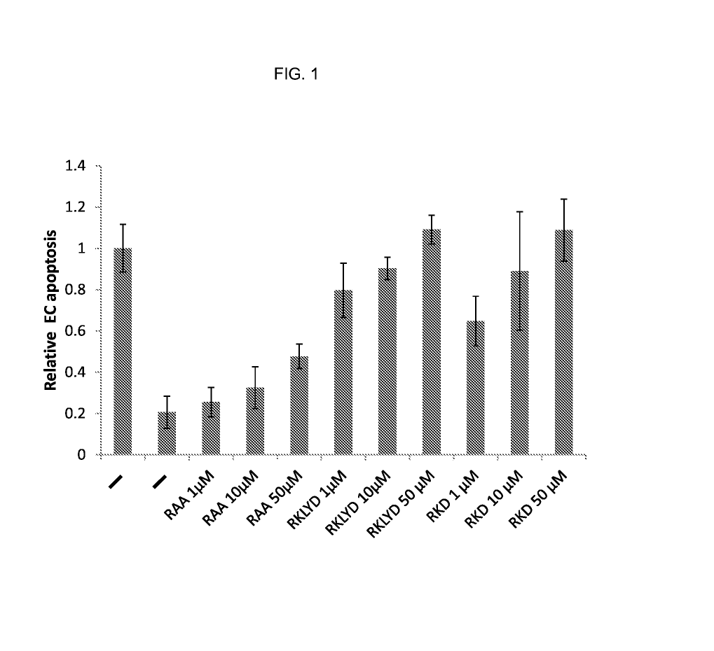

FIG. 1 Cyclic RKD (cRKD) peptide induces ECs apoptosis in a dose-dependent manner. “RKLYD” is a pro-apoptotic peptide derived from human plasminogen kringle 5 which is used as a control. “RKD” is equally potent comparing to “RKLYD”. “RAA” is a negative control peptide;

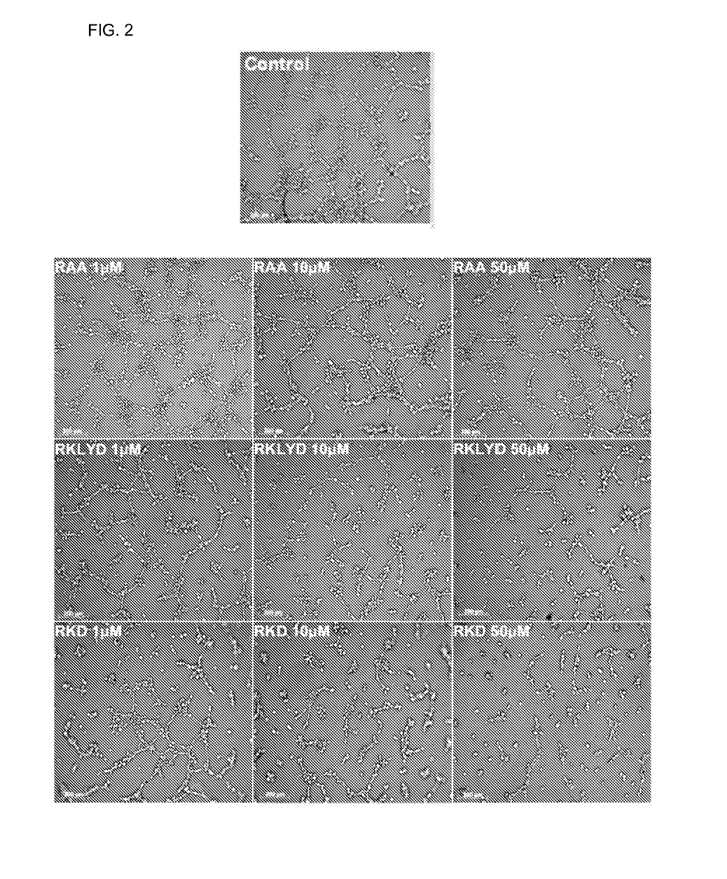

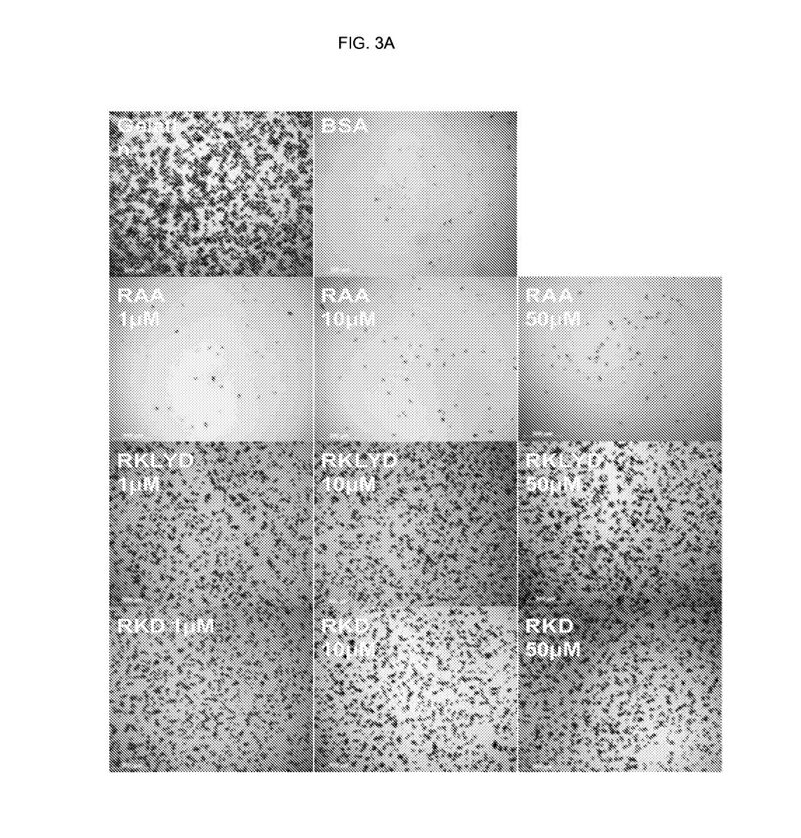

FIG. 2 cRKD peptide inhibits EC tube formation in a dose-dependent manner. “RKLYD” is an anti-angiogenic peptide derived from human plasminogen kringle 5 which is used as a control. “RAA” is a negative control peptide;

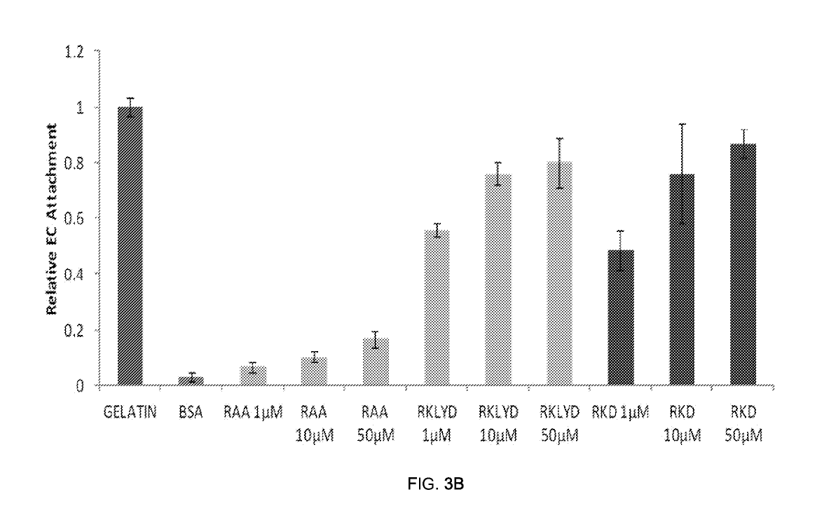

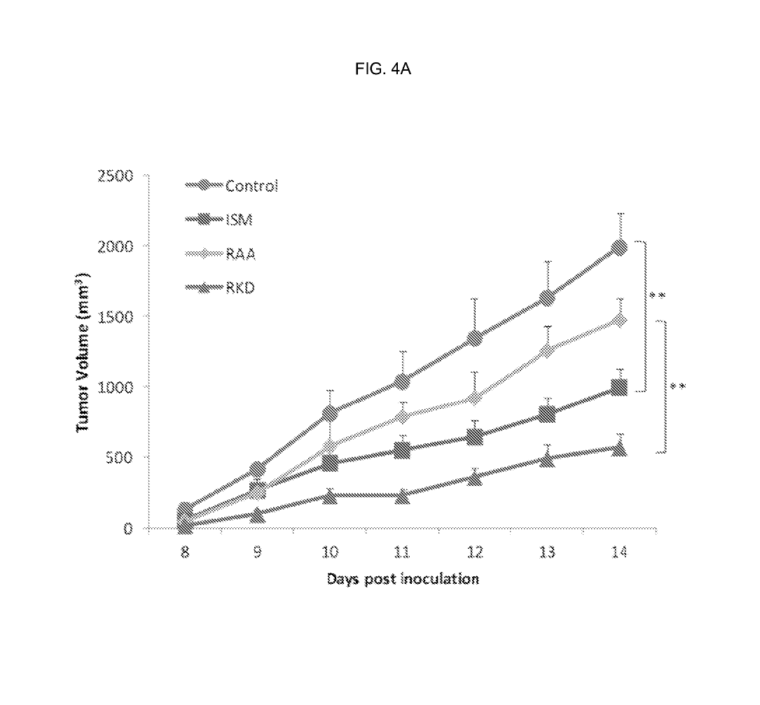

FIG. 3 cRKD peptide mediates EC adhesion in a dose-dependent manner. A. Representative photos of ECs adhered to peptide-coated surface. B. Quantification of the adhered ECs;

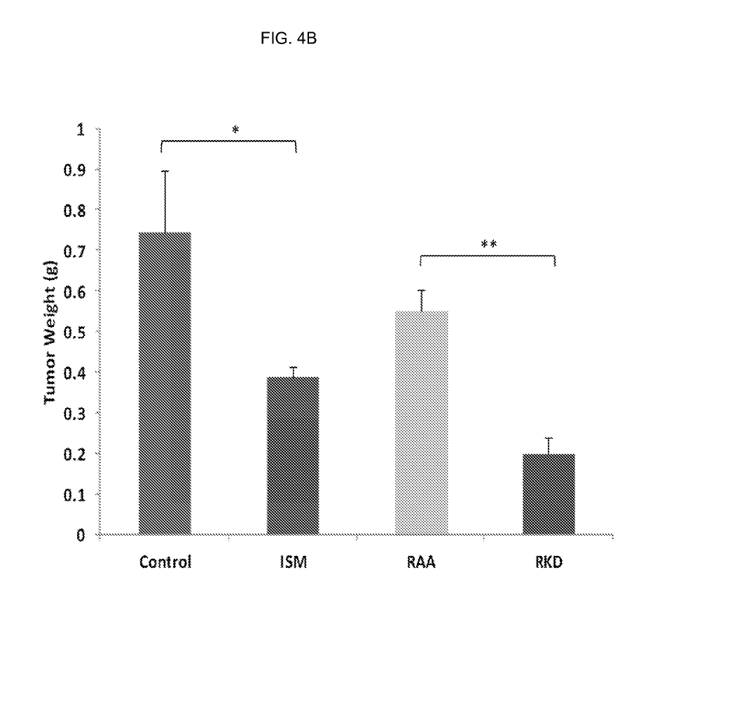

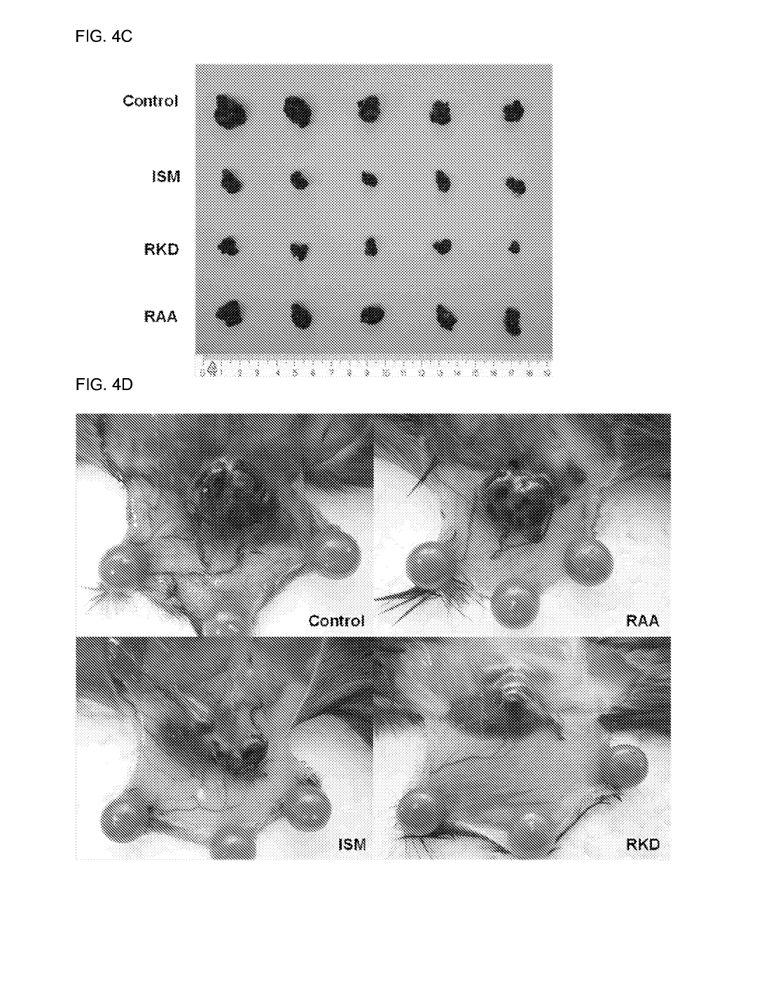

FIG. 4 Recombinant ISM and ISM derived cRKD peptide inhibit B16F10 tumour growth in vivo. A. Tumour growth curve in mice. X-axis represents the days after 5×105 tumour cell inoculation. Groups consisted of control mice receiving no treatment, recombinant ISM, cRKD peptide and its control-cyclic RAA peptide, given in six doses of 250 μg/mouse through tail vein injection on days 0, 2, 4, 6, 8, and 10 (5 mice in each group, each mice with one tumour). **: p<0.01,*: p<0.05, n=5. B. Tumour weight at the end of the experiment (14 days after tumour cell inoculation). C. Recombinant ISM and cRKD peptide inhibits B16F10 tumour growth. Photos of dissected tumours at the end of the experiment; D. Recombinant ISM protein and cRKD peptide show a reduced vascularization compared to control and cyclic RAA peptide;

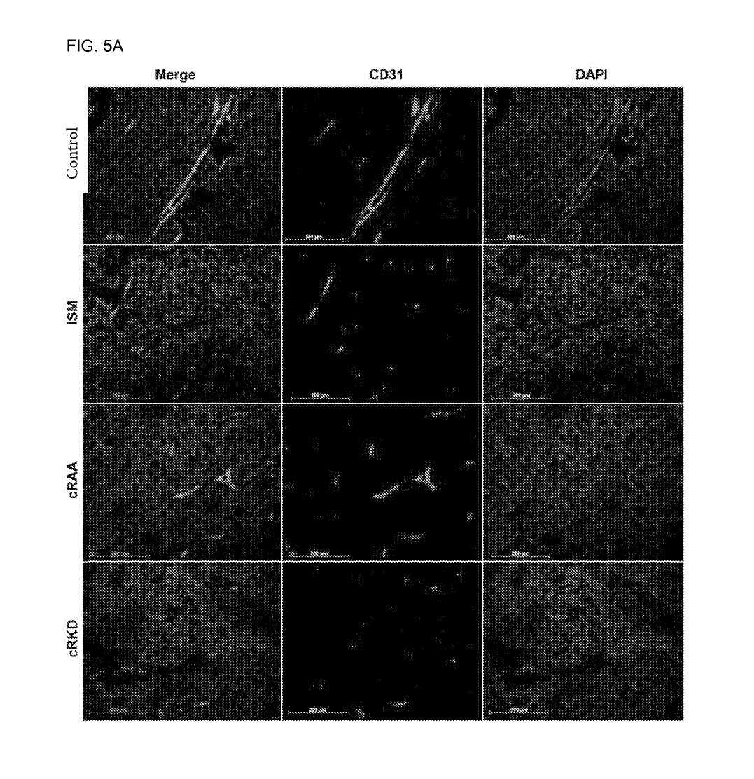

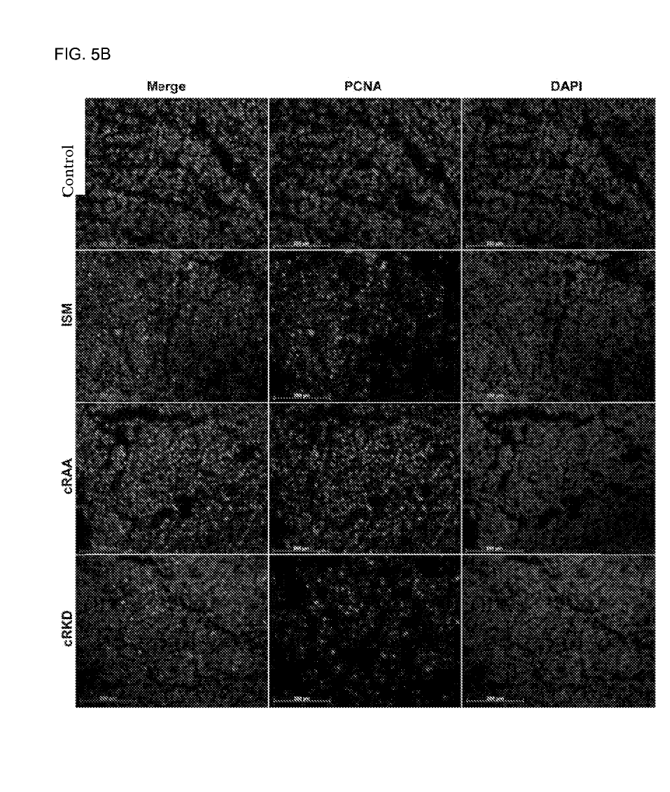

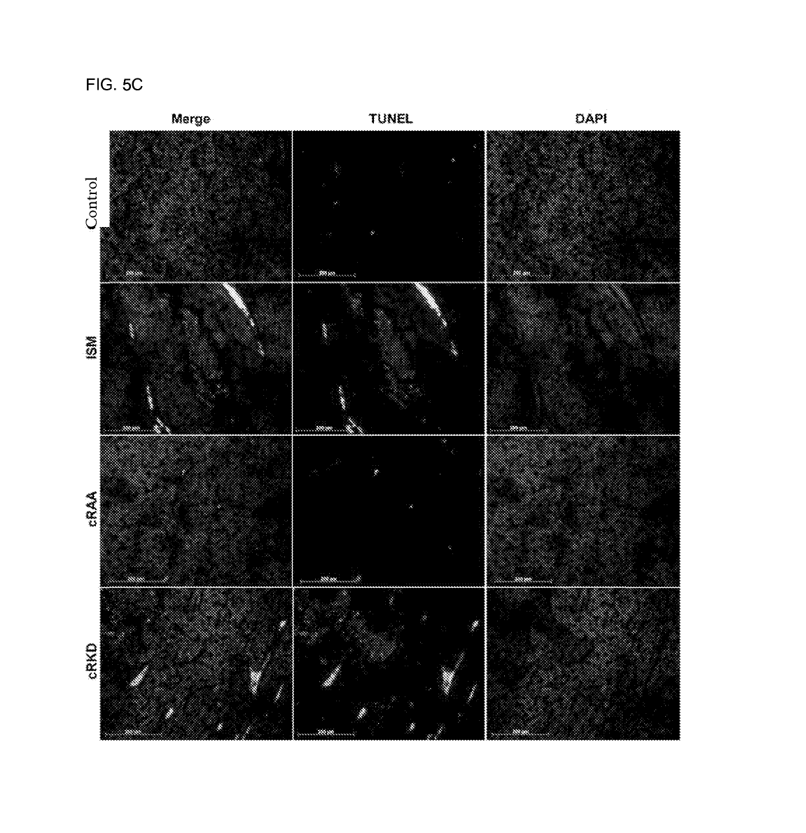

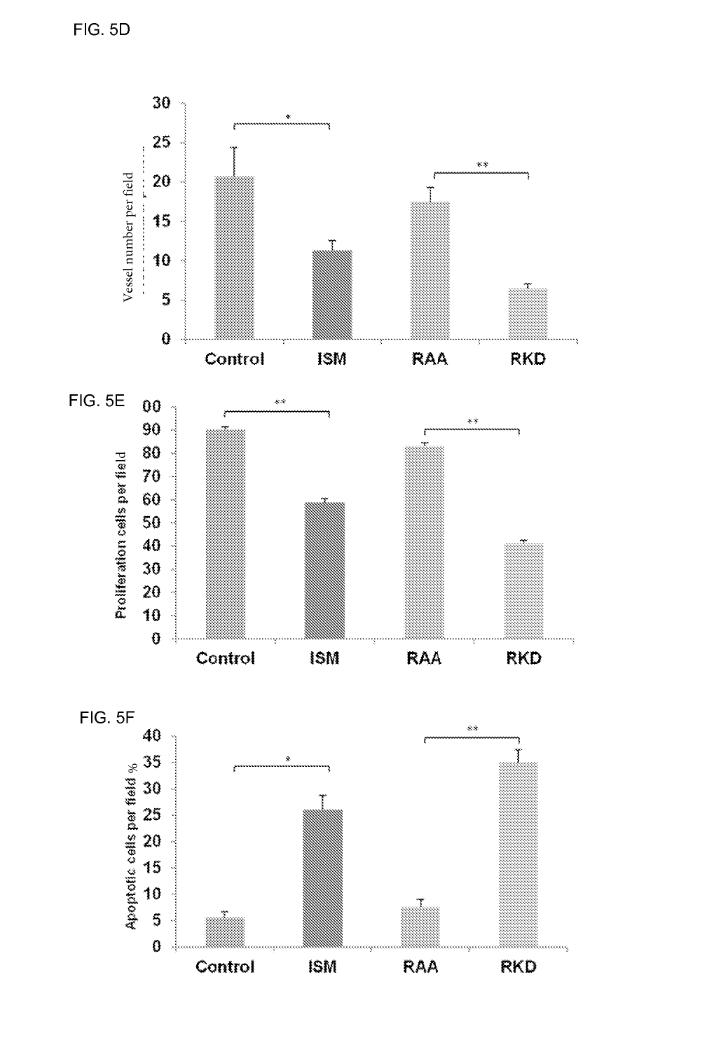

FIG. 5 Recombinant ISM and ISM derived cRKD peptide suppress tumor angiogenesis, tumor cell proliferation and induce tumor cell apoptosis. Paraffin sections of tumors from mice groups treated by control, ISM, cRAA and cRKD peptides were probed for microvessel density, tumor cell proliferation and apoptosis through immunofluoresence staining using anti-CD31 (A), anti-PCNA (B) and TUNEL staining (C) respectively. Representative photos are shown. (D) Quantification of microvessel density. Microvessel density is the number of microvessels per microscopic field. (E) Quantification of cell proliferation in tumor sample. Cell proliferation is presented as the percentage of PCNA positive cells out of the total number of cells (DAPI positive cells) in the microscopic field. (F) Quantification of apoptosis in tumor. Apoptosis is quantified as the percentage of TUNEL positive cells out of the total number of cells (DAPI positive cells) in the microscopic field. Plots represent the mean of 3 fields per section, 3 sections per tumor and 2 tumors per group (±SEM). ** P≦0.01, * P≦0.05;

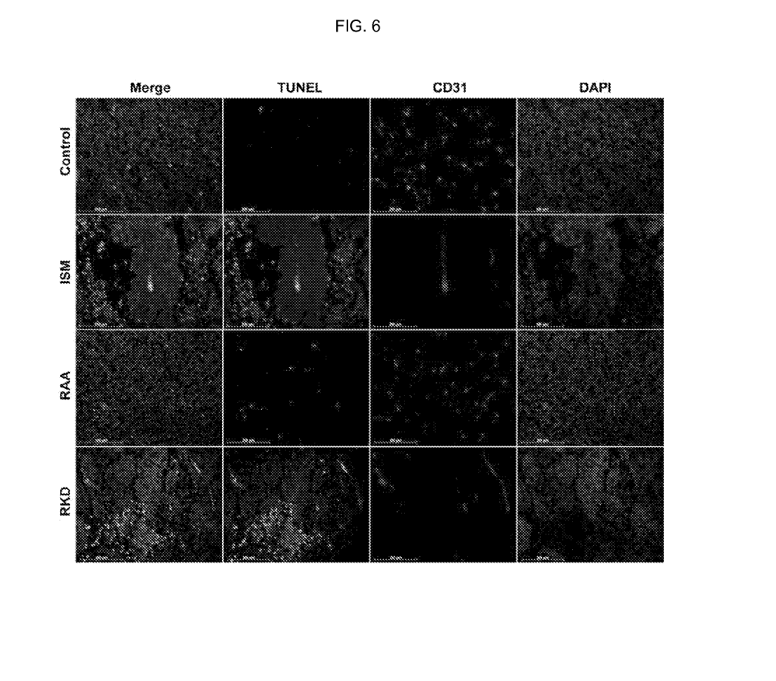

FIG. 6 Recombinant ISM and ISM derived cRKD peptide induce apoptosis of both tumor cell and tumor endothelial cells (ECs). Paraffin sections of tumors from mice groups treated by control, ISM, cRAA and cRKD peptides were probed for both microvessel and apoptosis through immunofluoresence staining using anti-CD31 (red) and TUNEL (green) double staining. Nuclei were counter stained by DAPI (blue). Representative photos are shown;

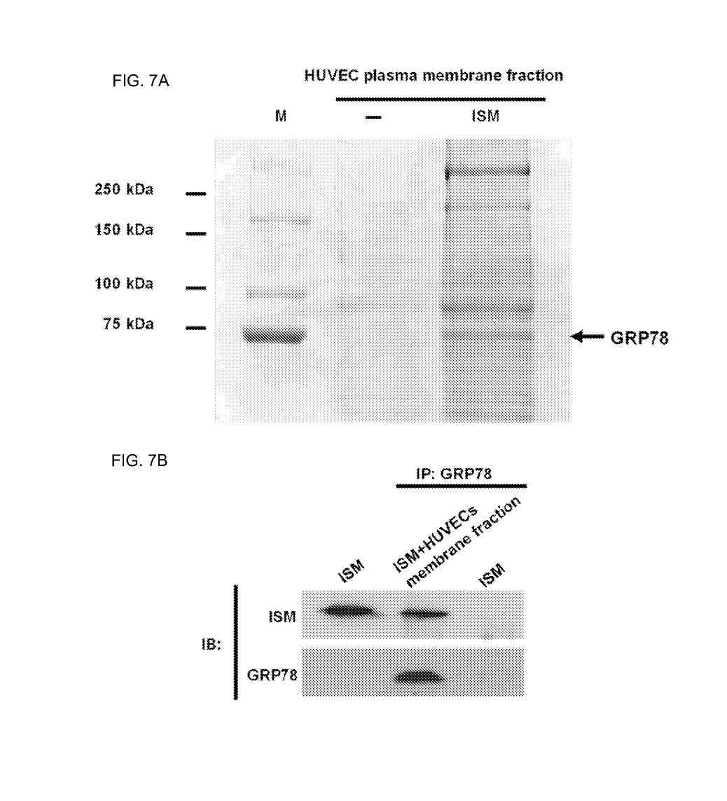

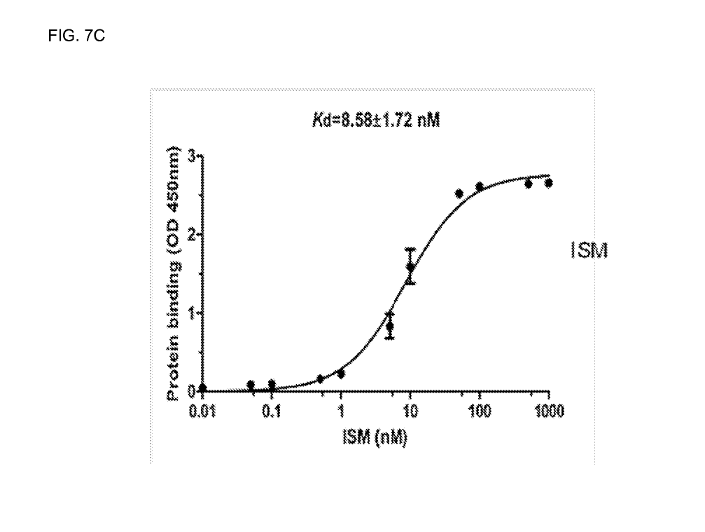

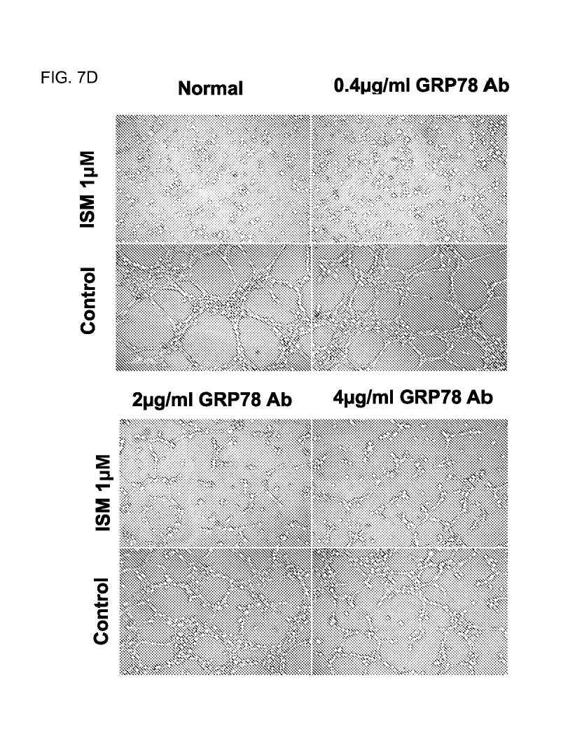

FIG. 7 GRP78 is a high affinity cell-surface receptor for ISM. A. ISM interacts with GRP78 on endothelial plasma membrane shown by pull-down experiment. Pull-down was accomplished by anti-His tag antibody which interact with recombinant His-tagged ISM. B. Co-imunoprecipitation demonstrates ISM interacts directly with GRP78. Purified recombinant ISM and GRP78 are incubated together in vivo and precipitated with anti-GRP78 antibody with protein A agarose beads. Immunoblot was then performed with anti-ISM antibody. C. ISM binds GRP78 with high affinity. D. GRP78 mediates the anti-angiogenic activity of ISM. Anti-GRP78 antibody does-dependently blocked the anti-angiogenic activity of ISM;

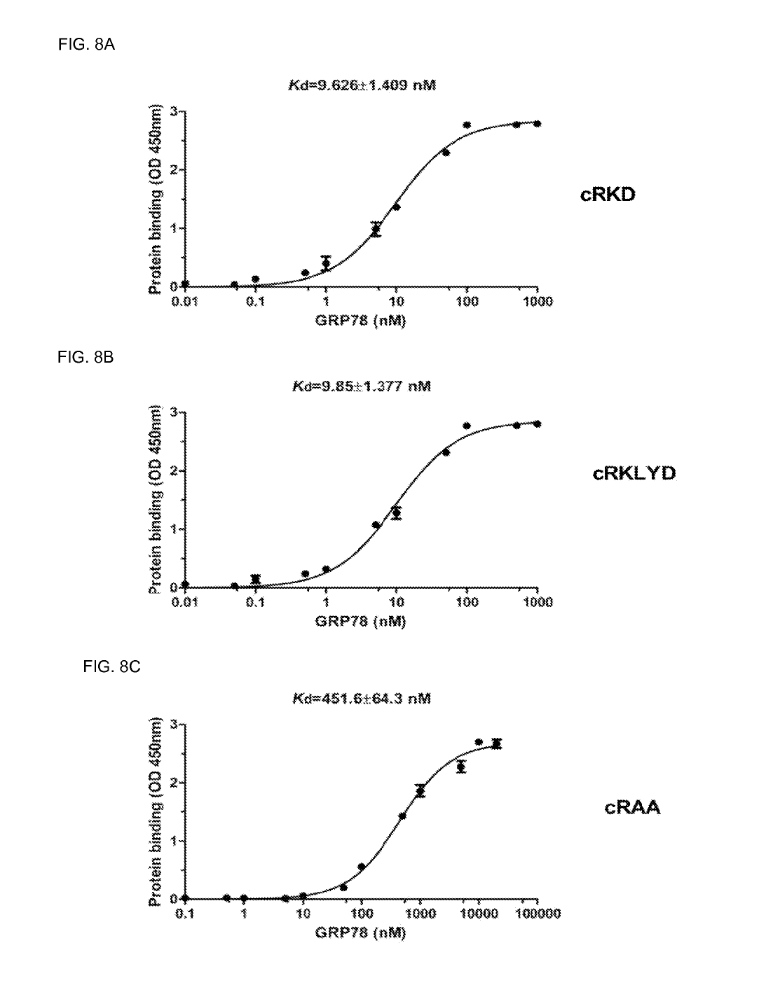

FIG. 8 cRKD peptide binds to GRP78 at high affinity. A. Binding affinity between cRKD and GRP78. n=3. B. Binding affinity between a known GRP78 ligand cyclic RKLYD peptide (cRKLYD) and GRP78. n=3. C. Binding affinity between control cRAA peptide and GRP78. n=3. Dose-response curve was generated and analyzed using GraphPad Prism software;

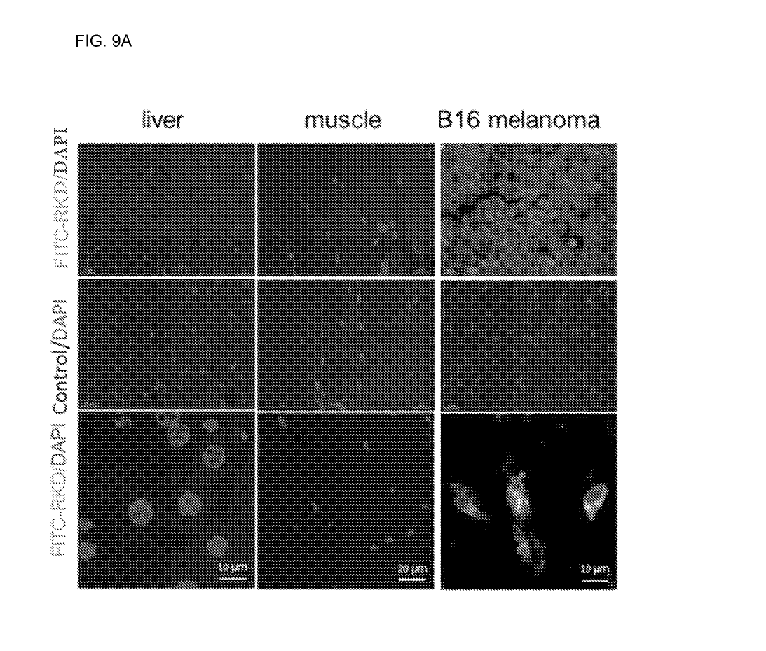

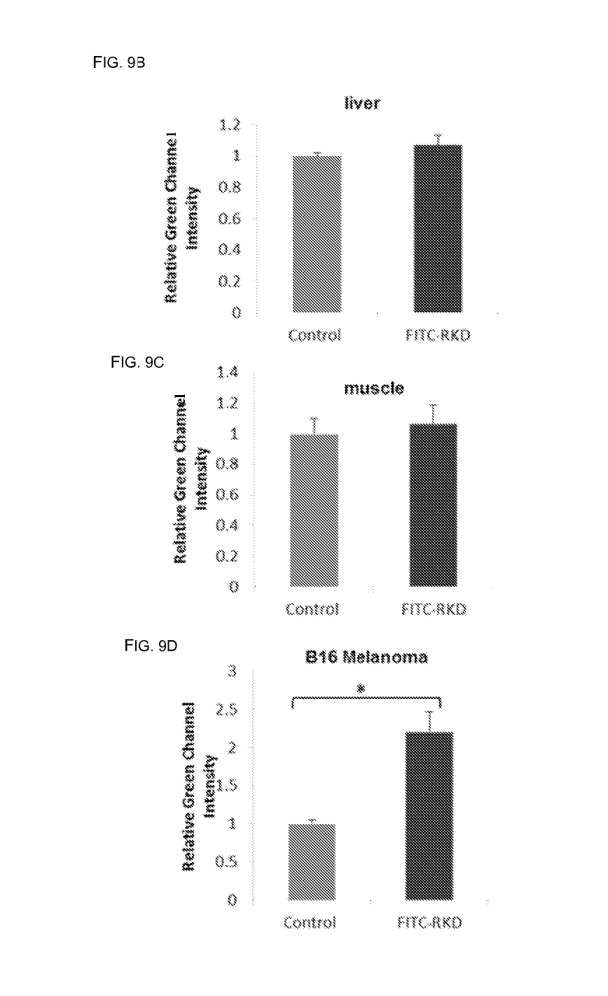

FIG. 9 FITC labelled cRKD (FITC-RKD) targets to subcutaneous B16 tumor in mice when delivered intravenously. Control samples were from tumor bearing mice that did not receive any FITC-RKD injection to show background fluorescent signal in tissues. The bottom row of A is higher magnifications of top row indicating that FITC-RKD is localized mainly in the cytosol of tumor cells;

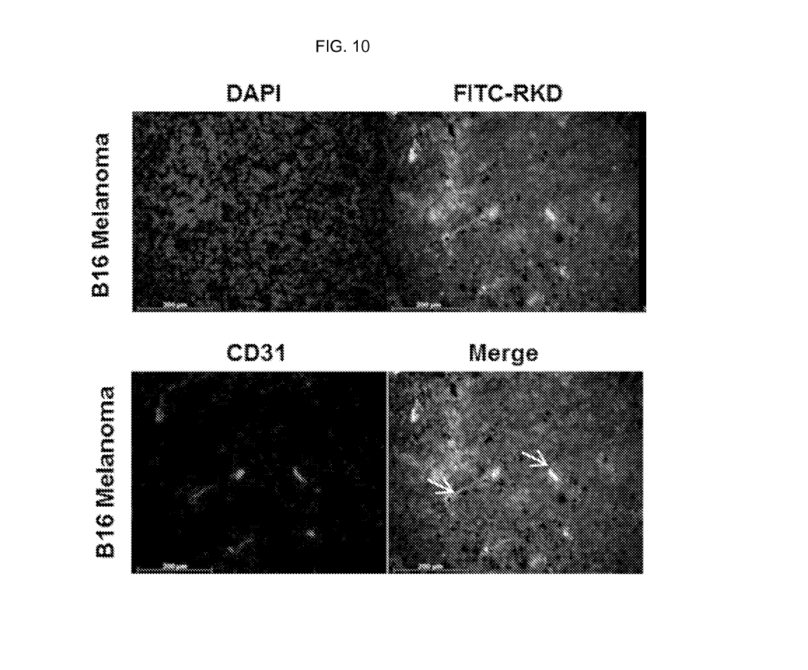

FIG. 10 FITC-RKD targets both the B16 melanoma tumor cells and tumor ECs when delivered intravenously;

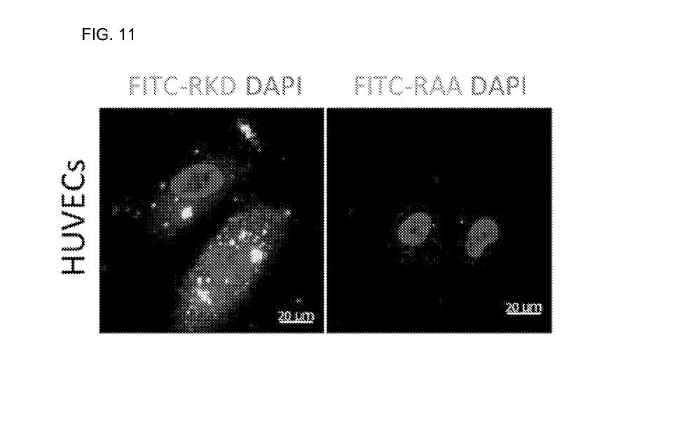

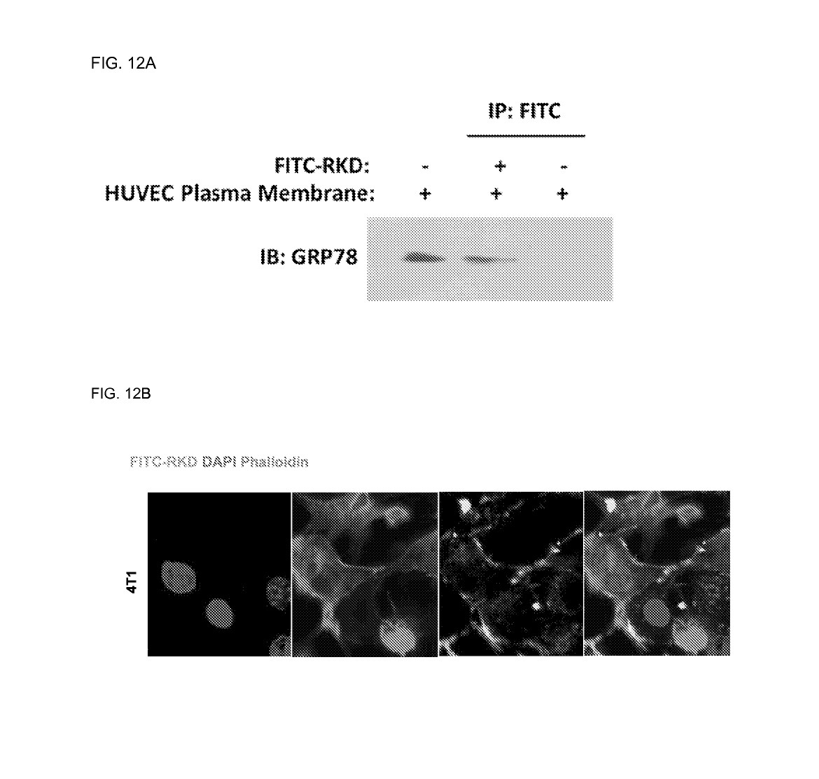

FIG. 11 FITC-RKD peptide enters cultured HUVECs likely by endocytosis;

FIG. 12a FITC-RKD peptide binds to cell-surface GRP78. FITC-RKD binds cell-surface GRP78 from the plasma membrane fraction of HUVECs as demonstrated by co-immunoprecipitation; FIG. 12b FITC-RKD binds 4T1 breast cancer cell surface in vitro, likely by binding to cell-surface GRP78. 4T1 breast carcinoma cells were treated with FITC-RKD (green) 24 h. Cells were fixed and stained by Phalloidin (red) to represent the cell outline. Nuclei were counter stained by DAPI (blue).

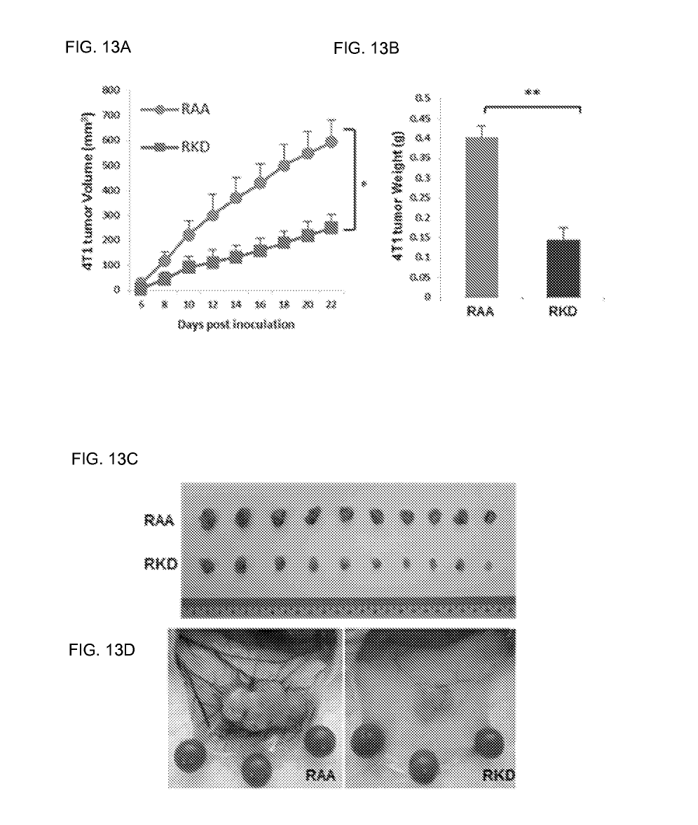

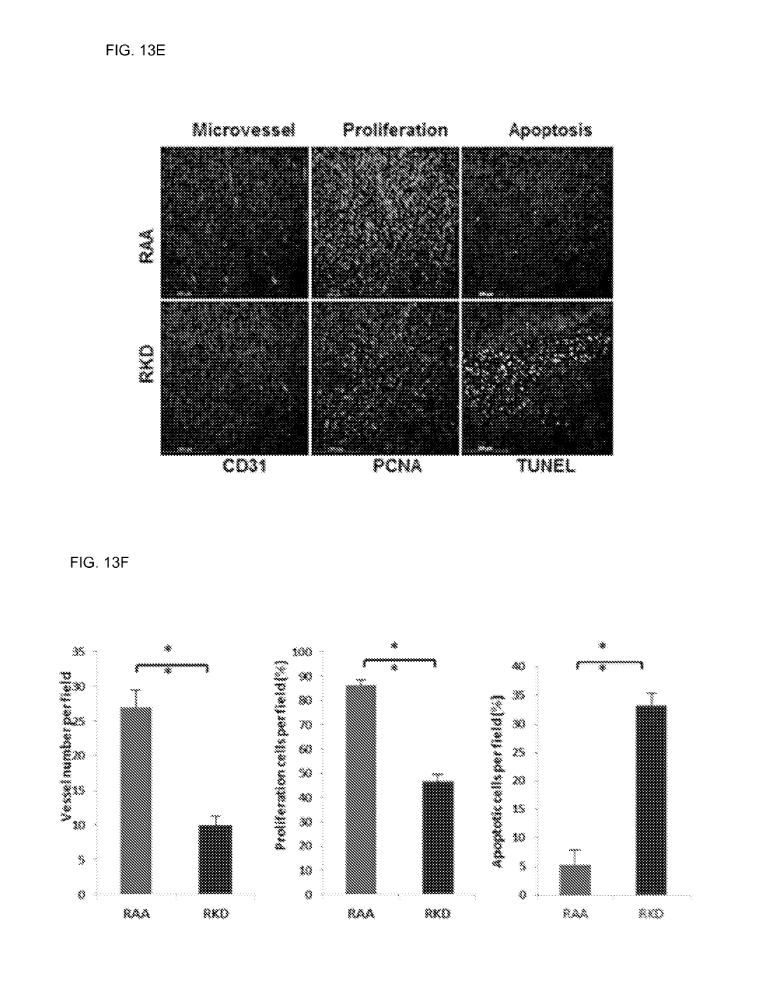

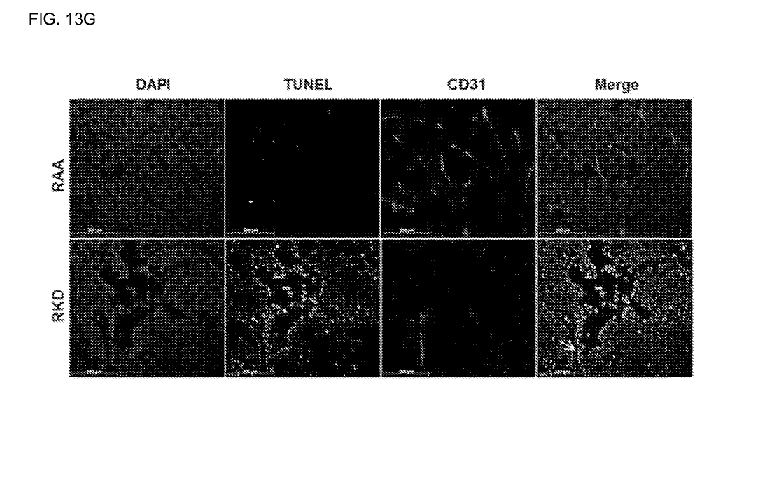

FIG. 13 cRKD peptide inhibits subcutaneous 4T1 breast carcinoma growth in mice when delivered intravenously. (a) 4T1 tumor growth curve in mice. X-axis represents the days after inoculation of 1 million tumor cells. Groups consisted of mice receiving 250 μg cRAA peptide or cRKD peptide through tail vein injection every other day from day 0 (date of inoculation) to 22. N=10. (b) Tumor weight at the end of the experiment (day 22). (c) Dissected tumors at the end of experiment. (d) cRKD peptide treated tumors showed a reduced vascularization compared to control. (e) cRKD peptide suppressed tumor angiogenesis, proliferation and induced apoptosis in 4T1 tumor. Paraffin sections of 4T1 tumors were probed for microvascular density (MVD), tumor cell proliferation and apoptosis through IF using anti-CD31, anti-PCNA and TUNEL staining respectively. (f) Quantification of MVD, cell proliferation and apoptosis. Plots represent the mean of 3 fields per section, 3 sections per tumor and 2 tumors per group. **P<0.01. Error bars denote SEM. (g) cRKD peptide induces apoptosis of both cancer cells and cancer ECs in 4T1 breast carcinoma. Double immunofluorescence staining using anti-CD31 (red) and TUNEL (green) is shown. Nuclei were counter stained by DAPI (blue). Representative photos are shown. Apoptotic EC is indicated by white arrow.

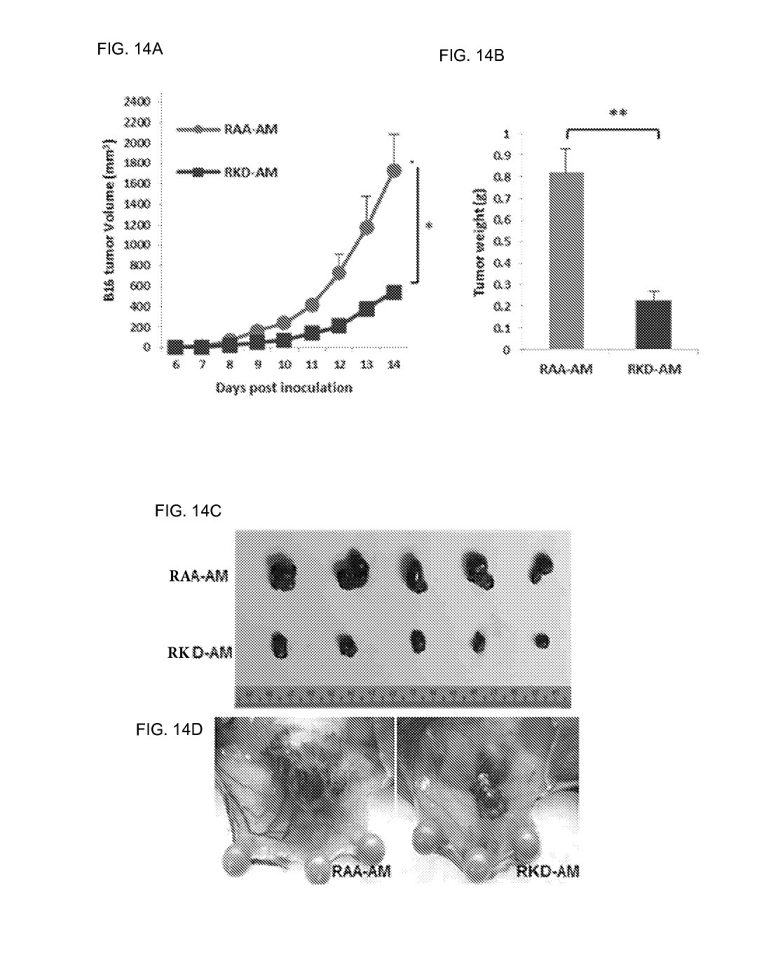

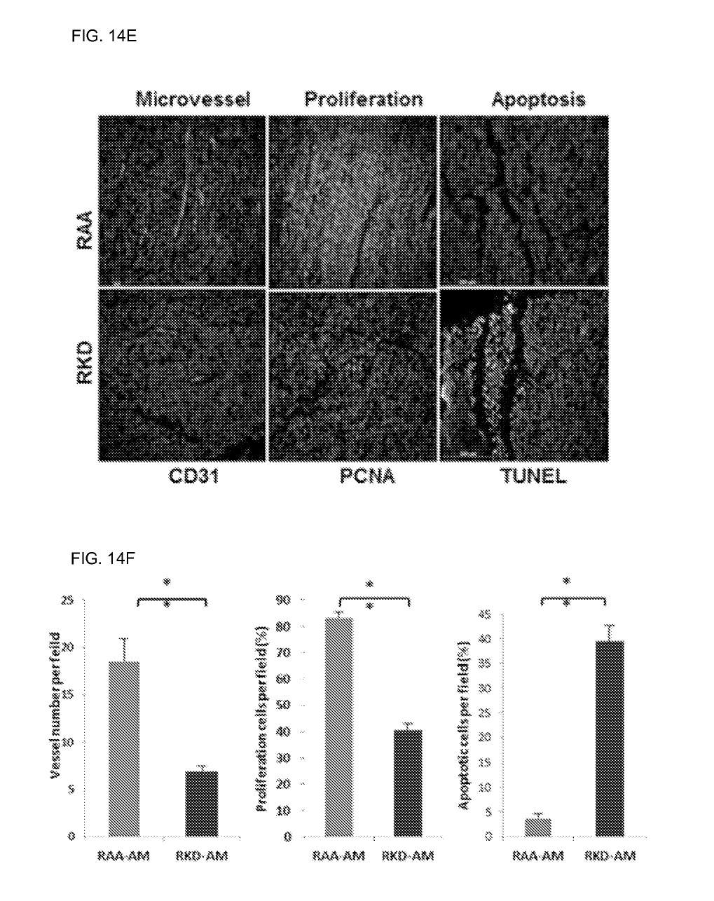

FIG. 14 Amide bond-cyclized RKD (RKD-AM) peptide inhibits subcutaneous B16 melanoma growth in mice when delivered intravenously; peptide treatment schedule was similar to that described in FIG. 4.

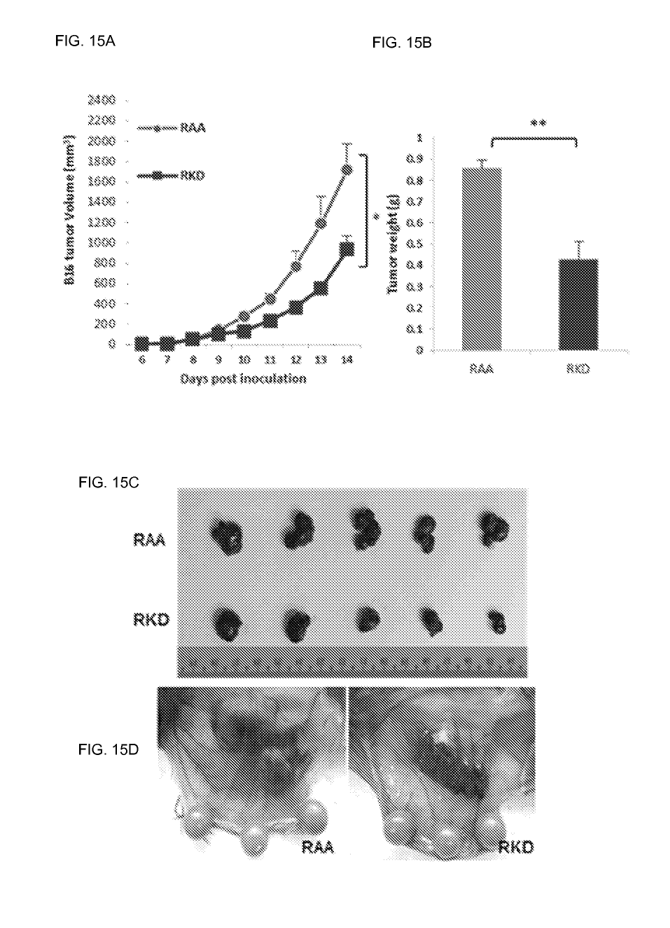

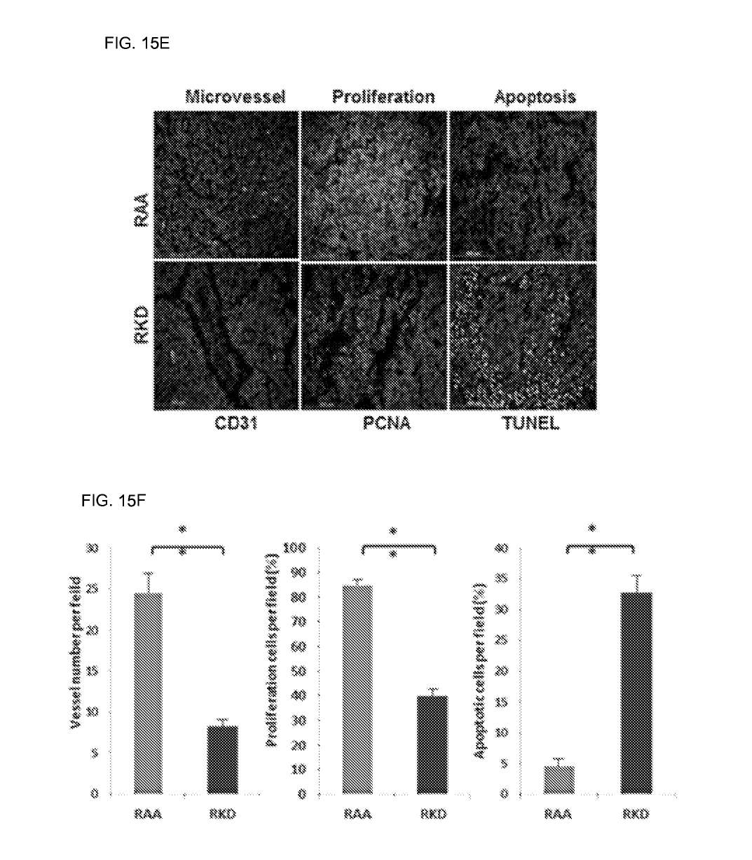

FIG. 15 cRKD peptide inhibits pre-established subcutaneous B16 melanoma growth in mice when delivered intravenously; (a) B16 tumor growth curve in mice. X-axis represents the days after inoculation of 5×10⁵ tumor cells. Groups consisted of mice receiving cRAA and cRKD given in six doses of 250 μg through tail vein injection on days 8, 9, 10, 11, 12 and 13 (5 mice in each group, each mice with one tumor). (b) Tumor weight at the end of the experiment (day 14). (c) Dissected tumors at the end of experiment. (d) cRKD peptide treated tumors showed a reduced vascularization compared to control. (e) cRKD peptide suppressed tumor angiogenesis, proliferation and induced apoptosis in B16 tumor. Paraffin sections of B16 tumors were probed for microvascular density (MVD), tumor cell proliferation and apoptosis through immunofluoresence staining using anti-CD31, anti-PCNA and TUNEL staining respectively. (f) Quantification of MVD, cell proliferation and apoptosis. Plots represent the mean of 3 fields per section, 3 sections per tumor and 2 tumors per group. **P<0.01. Error bars denote SEM.

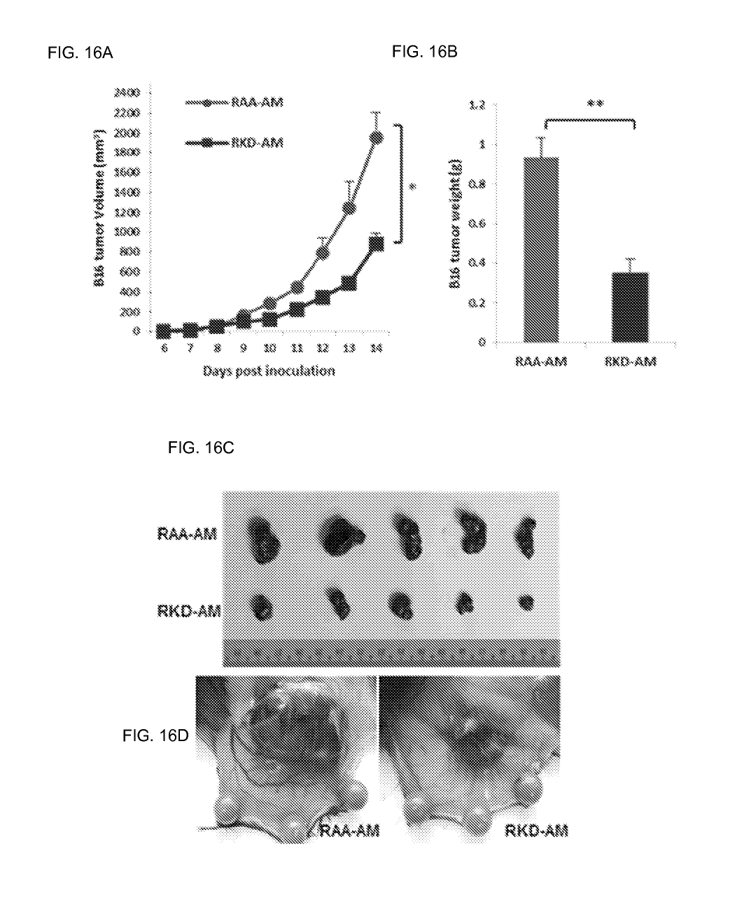

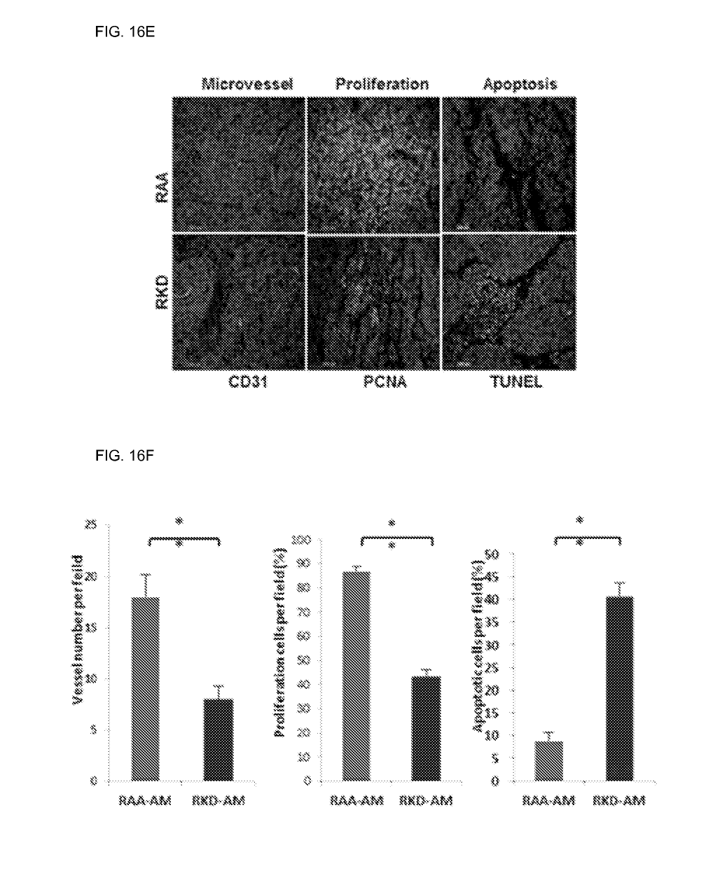

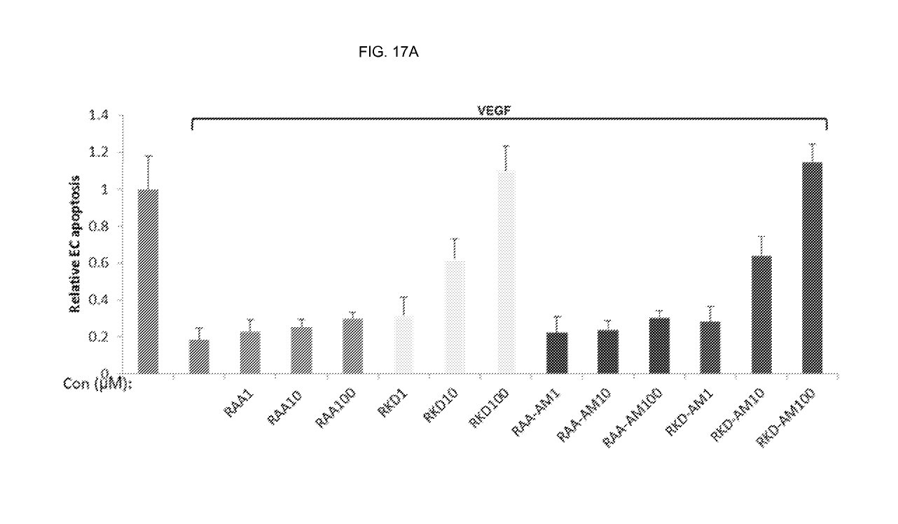

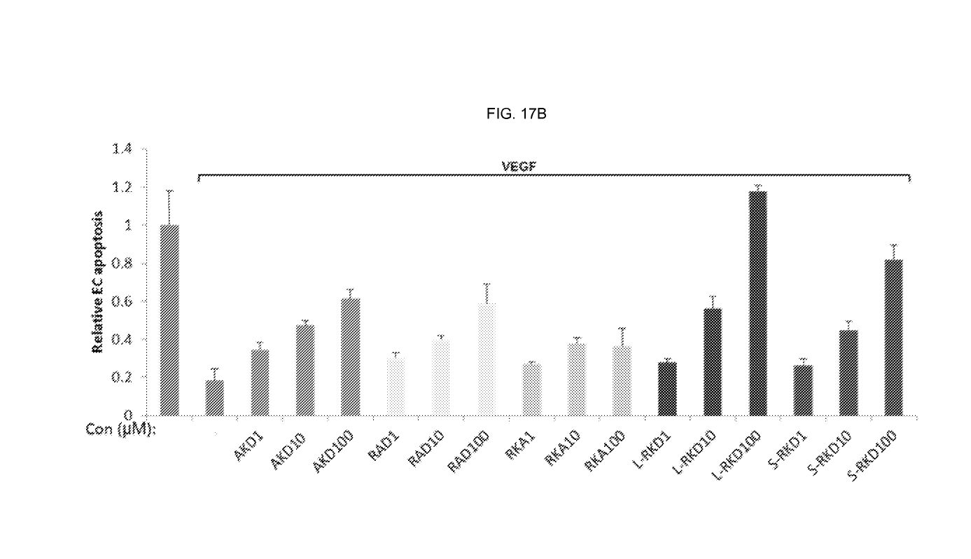

FIG. 16 RKD-AM peptide inhibits pre-established subcutaneous B16 melanoma growth in mice when delivered intravenously; treatment schedule similar to that described in FIG. 15; and

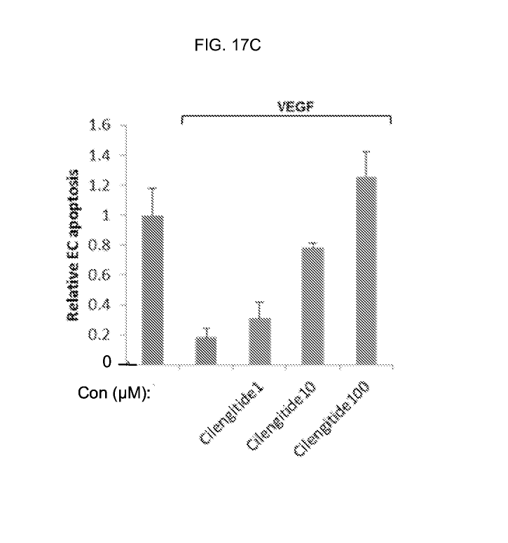

FIG. 17 Peptide induction of apoptosis in cultured ECs. The number 1, 10, and 100 that follow each peptide name indicate the peptide concentration used at 1 μM, 10 μM and 100 μM.

### MATERIALS & METHODS

### Tube Formation Assay

HUVECs (1×10⁴) were pre-treated with peptides of various concentrations for 30 min before being plated onto the polymerized Matrigel (Millipore, USA) in 15-well μ-slide (Ibidi, Germany). After 4-6 h, capillary network was documented using Zeiss Axiovert200 inverted microscope (Maple Grove, Minn., USA). Tube length was quantified by measuring the length of branches in representative fields using ImageJ software. Plots represented the mean of 3 wells of tubular length in 15-well μ-slide (t SEM).

### Apoptosis Assay

ECs (2×10⁴ cells per well) in 96-well plate were starved in 2% FBS basal CSC medium for 3 h. Cells were then incubated with peptides of various concentrations and 15 ng/ml VEGF for 24 h. Apoptosis was detected by measuring cytosolic oligonucleosome-bound DNA using a Cell Death ELISA kit purchased from Roche (USA). Plots represented the mean of 3 wells of apoptotic cells in 96 well plate (±SEM).

### Cell Attachment Assay

96-well plates were coated with 50 μl of 1 μM ISM at 4° C. overnight. Nonspecific binding sites were blocked with 1% BSA for 2 h at 37° C. ECs were harvested and incubated in CSC basal medium containing 4 μg/ml neutralizing antibodies or control IgG for 30 min. 2□104 cells were plated to each well and allowed to attach for 60 min at 37° C. Attached cells were fixed by 10% formalin and stained with 0.2% crystal violet. Absorbed crystal violet was extracted by 10% acetic acid and quantified by measuring the absorbance of eluted dye at 595 nm with a microplate reader.

### Mouse Tumorigenesis

Adult 8-week-old female C57BL/6J mice were used in this study. Animal care and experimentation was carried out under the institutional guidelines issued by the local institutional animal care and use committee (IACUC; protocol 066/12). 5×10⁵ B16F10 cells in 0.1 ml PBS were injected subcutaneously into the dorsal left flank of the mouse. Twenty mice were assigned into 4 different groups consisted of control mice receiving no treatment, recombinant ISM, cyclic RKD peptide and its peptide control-cyclic RAA peptide, given in six doses of 250 μg through tail vein injection on days 0, 2, 4, 6, 8, and 10 (5 mice in each group, each mice with one tumor). Health status of the mice was monitored over two weeks and visible tumor size were measured daily by calipers after 8 days until the end point (day 14). Tumor volume was calculated using the following formula: 0.52×length×(width)² mm³, where the largest dimension of the tumor is considered as the length and the width is the perpendicular dimension. On day 14, mice were sacrificed, and their dorsal flank was opened and photographed to exam the vascularization. Then tumors were excised and weighed followed by either fixing in 4% paraformaldehyde for sectioning or snap-freezing in liquid nitrogen for further analysis.

### Pull Down Assay

His-tagged ISM and its binding partners from plasma membrane extract were pulled down by Epitope Tag Protein Isolation Kit with separation columns purchased from μMACS (Bergisch Gladbach, Germany). For searching potential binding partners of ISM, eluants were subjected to SDS-PAGE and stained with coomassie blue. Excised bands were identified by MALDI-TOF-TOF mass spectrometry analysis.

### Determination of Binding Affinity Between Cyclic Peptide and GRP78 by ELISA

ELISA solid phase-binding assay was used to determine the dissociation constant between ISM or cyclic peptide and GRP78 as previously described with minor modification (Nishiuchi, Takagi et al. 2006). 96-well plates were coated with 50 μl of 10 nM cyclic peptide at 4° C. overnight. Nonspecific binding sites were blocked with 1% BSA for 2 h at room temperature. Increasing concentrations of GRP78 were incubated with immobilized cyclic peptide. After washing, the amount of bound GRP78 was quantified by ELISA. The value for K_{d} was calculated by GraphPad Prism software.

### Isolation of Plasma Membrane Fractions

The membrane fraction of HUVECs was isolated by using Mem-PER Eukaryotic Membrane Protein Extraction Kit which was also obtained from Pierce (Rockford, Ill., USA).

### Co-Immunoprecipitation

1 μg antibodies of GRP78 were pre-immobilized on 20 μl protein A/G agarose beads from Santa Cruz (Santa Cruz, Calif., USA) for 1 h at room temperature. Unbound antibodies were washed out by 100 mM Tris-Hcl (pH 8.0). Then 1 μg ISM and 1 μg GRP78 (or 1 mg membrane fraction protein lysates containing GRP78) were incubated with GRP78 antibody mounted beads for 4 h at 4° C. After removing the unbound proteins, precipitates were resolved by SDS-PAGE and analyzed for the presence of ISM, GRP78 or AACs by Western blotting.

### Immunohistochemistry

Paraffin-embedded tissue sections of 5 μm were deparaffinized by heating at 60° C. for 7 minutes before deparaffinizing in histoclear followed by the rehydration in graded series of alcohol (100%, 90%, 80%, and 70% ethanol in ddH2O) and finally in PBS for 5 minutes. Antigen retrieval was done in a bench-top 2100-Retriever according to the protocol of the manufacturer (Electron Microscopy Sciences, Hatfield, Pa.). The sections were blocked with 3% bovine serum albumin (BSA) in a humidified chamber for 1 hour at room temperature followed by overnight incubation with the anti-CD31 (Santa Cruz Biotechnology, Santa Cruz, Calif.), anti-PCNA antibody (Santa Cruz Biotechnology), anti-GRP78 antibody (Santa Cruz Biotechnology), or anti-His tag antibody (Santa Cruz Biotechnology) at 4° C. The following day, unbound antibody was washed with 1×PBS containing 0.1% Tween-20 (1×PBST) and incubated with the corresponding Alexafluor 568 secondary antibodies (Life Technologies) for 1.5 hours at 37° C. Sections were rinsed again and incubated for 10 minutes with DAPI at 1 μg/mL to visualize the DNA in the cell nucleus. Images were obtained using fluorescence microscope fitted with a digital camera (Zeiss Axiovert 200 or Zeiss LSM 510 Meta). Terminal deoxynucleotidyl transferase dUTP nick end labelling (TUNEL) was performed on the mouse tumor sections for the detection of apoptotic cells using an in situ cell death detection kit (Roche).

### Statistical Analysis

Data were expressed as standard errors of the mean (±SEM). Statistical significance was determined using Student's t-test. *P<0.05; **P<0.01.

### EXAMPLE 1

Cyclic CRKDC peptide (cRKD) induces potent endothelial cell apoptosis in a dose-dependent manner.

The cRKD peptide potently induced apoptosis of cultured ECs in the presence of VEGF similar to ISM protein or the cRKLYD peptide derived from kringle 5 of human plasminogen (FIG. 1) ((Davidson, Haskell et al. 2005, Xiang, Ke et al. 2011). In comparison, the mutant cRAA peptide significantly lost this pro-apoptotic activity with almost no activity at 1 μM. The potency of cRKD is similar to that of cRKLYD with cRKS somewhat less potent at lower concentration.

### EXAMPLE 2

### cRKD Peptide Inhibits EC Tube Formation on Matrigel

cRKD peptide does-dependently inhibited EC tube-like structure formation on Matrigel similar to cRKLYD while cRAA lost this activity (FIG. 2). At 1 μM, cRAA has no anti-angiogenic activity while both cRKD and cRKLYD inhibited angiogenesis with similar potency.

### EXAMPLE 3

### cRKD Mediate EC Adhesion

When cRKD is coated on plastic surface, it mediated EC attachment and adhesion similar to the extracellular matrix cell adhesion molecule Geletin (a form of collagen) or cRKLYD. In comparison, cRAA has lost the ability to mediate EC adhesion (FIGS. 3, A and B).

### EXAMPLE 4

### Systemic Delivery of Recombinant ISM and cRKD Peptide Potently Suppressed Melanoma Growth in Mice

When cRKD peptide is delivered systemically through intravenous injection to mice implanted with subcutaneous B16F10 melanoma at 250 μg/mouse/every 2 days, it significantly suppressed tumor growth comparing to cRAA peptide treated mice (FIGS. 4, A, B and C). Similarly, recombinant mouse ISM protein also suppressed tumor growth under similar conditions when compared to untreated control (FIGS. 4, A and B). Hence, it seems that cRKD peptide can function as an anti-angiogenic and anti-tumorigenic molecule in similar fashion as ISM protein. In addition, the blood vessels supplying the tumor are obviously reduced in cRKD treated mice (FIG. 4D).

Analyses of tumor tissue sections by fluorescent immunohistochemistry demonstrate an obvious reduction of tumor vessel density in cRKD and ISM treated tumors (FIG. 5A). At the same time, tumor cell proliferation is reduced while apoptosis in the tumor tissue indicated by TUNEL staining is significantly increased (FIG. 5, B, C). Quantitation of tumor vessel density indicated that cRKD suppressed tumor angiogenesis more than 3-fold while apoptosis in the tumor tissue increased more than 3-fold. Additionally, tumor cell proliferation was suppressed more than 2-fold (FIG. 5, D-F).

Double staining of tumor blood vessel ECs and apoptotic cells revealed that both tumor cells as well as tumor ECs became apoptotic when treated by cRKD or ISM comparing to controls (FIG. 6).

### EXAMPLE 5

### Cell-Surface GRP78 is a High Affinity Receptor for ISM

GRP78 is a stress response protein which is known to be translocated onto cell surface when overexpressed or under stress such as in cancer cells and cancer endothelial cells (Lee 2007, Ni, Zhang et al. 2011). Incubation of ISM with plasma membrane extract from HUVECs followed by pull-down with anti-ISM antibody identified GRP78 as a ISM binding protein (FIG. 7A). Co-immunoprecipitation experiment confirmed the ISM-GRP78 interaction using purified recombinant ISM and GRP78 proteins (FIG. 7B). Binding affinity between ISM and GRP78 is determined with a Kd of 8.6 nM (FIG. 7C). Anti-GRP78 antibody dose-dependently blocked the anti-angiogenic activity of ISM (FIG. 7D). Hence, GRP78 is a high affinity cell surface receptor for ISM.

### EXAMPLE 6

### cRKD Peptide Binds GRP78 with High Affinity

Using similar ELISA method, we determined the binding affinity between kRKD peptide and GRP78 using purified recombinant GRP78 protein. The peptide binds to GRP78 with a high affinity, with Kd=9.6 nM (FIG. 8A). This binding affinity is similar to ISM or cRKLYD (FIGS. 7C and 8B). In comparison, the cRAA peptide binds with a much lower affinity (about 50-fold less) (FIG. 8C), corresponding to a loss of anti-angiogenic activity.

Peptide cRKD (MW-600) binds GRP78 with high affinity (Kd=9.6 nM). The restricted and high level expression of cell-surface GRP78 on cancer cells and cancer blood vessel ECs projected that labelled cRKD peptide will selectively bind to cells in cancer, but not cells in normal organs. Indeed, FITC-labelled cRKD (FITC-RKD) preferentially labelled xenograft melanoma in mice when delivered intravenously (FIG. 9). Both tumor cells and tumor blood vessel endothelial cells are labeled by FITC-RKD (FIG. 10). FITC-RKD was also efficiently taking up by cultured HUVECs (human umbilical vein endothelial cells) much more efficiently comparing to FITC-labeled cRAA peptide (a mutant peptide control) (FIG. 11). Co-immunoprecipitation experiment using anti-FITC antibody indicated that FITC-RKD binds cell-surface GRP78 in the plasma membrane fraction of cultured HUVECs (FIG. 12). Hence, labeled cRKD can function as a imaging probe to reveal GRP78 level in cancer which can serve as a companion diagnostic/prognostic/predictive biomarker for cancer aggressiveness and chemoresistance.

Small peptides for receptor imaging are advantageous over proteins and antibodies. Peptides are small molecules and can rapidly diffuse into target tissue. They also clear rapidly from the blood and non-target tissues, resulting in high tumor-to-background ratios. Furthermore, peptides generally are non-immunogenic due to their small sizes. Radiolabelled peptides have been successfully used in clinical SPECT (Single-photon emission computed tomography) and PET (Positron Emission Tomography).

Thus, peptide cRKD has several favorable features allowing it to be a successful imaging probe:

(a) It is a small molecule, can rapidly diffuse to target tissue;
(b) It can be chemically synthesized in large quantity with consistent quality;
(c) Its target is on cell-surface, no need of cell penetration;
(d) It binds to its receptor with high affinity at single digit nM;
(e) It can preferentially home to tumor cells/tumor ECs and label xenograft tumor in mice.
(f) GRP78 is an important prognostic/predictive biomarker for cancer aggressiveness and chemoresistance. No imaging agent targeting GRP78 is currently available.

### EXAMPLE 7

cRKD also suppressed subcutaneous 4T1 breast cancer growth in syngeneic mice when delivered intravenously (FIG. 13). In addition to disulfide-bond cyclized peptide, amide-bond cyclized RKD peptide (RKD-AM) also suppressed B16 melanoma growth in mice (FIG. 14).

Both disulfide-bond linked and amide-bond linked RKD also suppressed pre-existing B16 melanoma growth in mice, similar to human cancers in clinical settings (FIGS. 15&16). Thus, cRKD has the potential to serve as anticancer drugs.

The importance of the core RKD amino acid sequence is demonstrated by mutational analyses of the cRKD peptide. As shown in FIG. 17, mutating any one of the three core residues lead to significant reduction of proapoptotic activity of cRKD peptide. Mutating KD simultaneously (RAA) completely destroyed the proapoptotic activity of RKD.

Furthermore, the fact that amide-bond cyclized RKD-AM peptide also induced EC apoptosis with similar potency as cRKD also confirmed the critical roles of the RKD sequence in conferring the proapoptotic function to this peptide. Additionally, longer cyclic peptides containing the RKD sequence such as S-RKD (Sequence ID. 22) and L-RKD (sequence ID. 23) which contain native ISM protein sequence surrounding the RKD motif also harbor similar proapoptotic function as cRKD. Hence, these peptides also have the potential to be anticancer drugs.

TABLE 1
Peptides tested in various biological assays

| | | | Molecular |
|---|---|---|---|
| Peptide | Peptide | | weight |
| name | sequence | Modification | (g/mol) |
| RKD | CRKDC | Disulfide | 621.74 |
| | | bridge: 1-5 | |
| RAA | CRAAC | Disulfide | 520.63 |
| | | bridge: 1-5 | |
| RKD-AM | CRKDC | Amide | 605.74 |
| | | cyclic: 1-5 | |
| RAA-AM | CRAAC | Amide | 504.63 |
| | | cyclic: 1-5 | |
| S-RKD | CKRKDFC | Disulfide | 897.09 |
| | | bridge: 1-7 | |
| L-RKD | CDRIKRK | Disulfide | 1867.2 |
| | DFRWKDC | bridge: 1-14 | |
| AKD | CAKDC | Disulfide | 536.63 |
| | | bridge: 1-5 | |
| RAD | CRADC | Disulfide | 564.64 |
| | | bridge: 1-5 | |
| RKA | CRKAC | Disulfide | 577.73 |
| | | bridge: 1-5 | |
| Cilen- | RGDfV | Amide | 588.66 |
| gitide | | cyclic: 1-5 | |
| FITC-RKD | FITC- | Disulfide | 1124.22 |
| | CRKDC | bridge: 1-5 | |
| FITC-RAA | FITC- | Disulfide | 1023.11 |
| | CRAAC | bridge:1-5 | |

### REFERENCES

Davidson, D. J., C. Haskell, S. Majest, A. Kherzai, D. A. Egan, K. A. Walter, A. Schneider, E. F. Gubbins, L. Solomon, Z. Chen, R. Lesniewski and J. Henkin (2005). “Kringle 5 of human plasminogen induces apoptosis of endothelial and tumor cells through surface-expressed glucose-regulated protein 78.” Cancer research 65(11): 4663-4672.
Lee, A. S. (2007). “GRP78 induction in cancer: therapeutic and prognostic implications.” Cancer Res 67(8): 3496-3499.
Ni, M., Y. Zhang and A. S. Lee (2011). “Beyond the endoplasmic reticulum: atypical GRP78 in cell viability, signalling and therapeutic targeting.” The Biochemical journal 434(2): 181-188.
Nishiuchi, R., J. Takagi, M. Hayashi, H. Ido, Y. Yagi, N. Sanzen, T. Tsuji, M. Yamada and K. Sekiguchi (2006). “Ligand-binding specificities of laminin-binding integrins: a comprehensive survey of laminin-integrin interactions using recombinant alpha3beta1, alpha6beta1, alpha7beta1 and alpha6beta4 integrins.” Matrix Biol 25(3): 189-197.
Xiang, W., Z. Ke, Y. Zhang, G. Ho-Yuet Cheng, I. D. Irwan, K. N. Sulochana, P. Potturi, Z. Wang, H. Yang, J. Wang, L. Zhuo, R. M. Kini and R. Ge (2011). “Isthmin is a novel secreted angiogenesis inhibitor that inhibits tumour growth in mice.” Journal of cellular and molecular medicine 15(2): 359-374.

1. An agent comprising a cyclic peptide comprising the amino acid motif argininelysine-aspartic acid [RKD] for use as a medicament.

2. The agent according to claim 1, wherein said peptide comprises the amino acid motif cysteine-arginine-lysine-aspartic acid-cysteine [SEQ ID NO: 5].

3. The agent according to claim 1, wherein said peptide consists of the amino acid motif cysteine-arginine-lysine-aspartic acid-cysteine.

4. The agent of claim 1, wherein the cyclic peptide includes more than one amino acid motif comprising the amino acid sequence RKD.

5. The agent of claim 1, wherein the cyclic peptide is pegylated.

6. A pharmaceutical composition comprising a cyclic peptide comprising the amino acid motif arginine-lysine-aspartic acid [RKD] and a pharmaceutically acceptable excipient and/or carrier.

7. The composition according to claim 6, wherein said composition comprises an additional, different therapeutic agent.

8. The composition according to claim 7, wherein said additional therapeutic agent is an anti-cancer agent.

9. The composition according to claim 7, wherein said additional therapeutic agent is a chemotherapeutic agent or an anti-angiogenic agent.

10. (canceled)

11. A method of treating a condition in a subject that would benefit from inhibition of excessive or abnormal angiogenesis, comprising:
administering the agent of claim 1 to the subject.

12. The method according to claim 11 wherein said condition is cancer, metastatic cancer, diabetes mellitus, diabetic retinopathy, diabetic nephropathy, rheumatoid arthritis, psoriasis.

13. (canceled)

14. The method according to claim 12, wherein said cancer comprises cells that express or over expresses glucose regulated protein 78 [GPR78].

15. The method according to claim 14, wherein said cancer is selected from the group consisting of: liver cancer, prostate cancer, skin cancer, melanoma cancer, breast cancer and colon cancer.

16-20. (canceled)

21. The method according to claim 11, wherein said condition is an eye related condition selected from the group: age related macular degeneration, neovascular glaucoma, corneal neovascularization [trachoma] and pterygium.

22. An imaging agent comprising a cyclic peptide comprising the amino acid motif arginine-lysine-aspartic acid [RKD].

23. The imaging agent according to claim 22, wherein said imaging agent comprises a fluorescence molecule or a radioisotope.

24. The imaging agent according to claim 23, wherein said fluorescence molecule is a fluorescent dye or a fluorescent protein.

25-26. (canceled)

27. A method of imaging a tumour, comprising:
i) administering the imaging agent of claim 22 to a subject; and
ii) detecting the imaging agent bound to GPR78 expressed by a tumour cell, tumor endothelial cell and/or cancer stem cell.

28. The method according to claim 27, wherein said method is single-photon emission computed tomography, positron emission tomography, or fluorescence microscopy.

29-37. (canceled)

38. The method of claim 11, wherein cells associated with said condition express or over express glucose regulated protein 78 [GPR78].

39. The composition according to claim 6, wherein said cyclic peptide:
comprises an amino acid sequence as set forth in SEQ ID NO: 1 or 20, or an amino acid sequence variant of SEQ ID NO: 1 or 20, wherein said variant is modified by addition, deletion or substitution of one or more amino acid residues and wherein said polypeptide has retained or enhanced binding to GPR78;
comprises amino acid sequences 289-452 of SEQ ID NO: 1 or 20; or
consists essentially of the amino acid sequence set forth in SEQ ID NO: 3.

40-41. (canceled)

42. The composition of claim 39, wherein said cyclic peptide is between 3 and 163 amino acids.

43. The composition according to claim 42 wherein said cyclic peptide is 5, 10, 20, 25, 30, 40, 50, 60, 70, 80, 90, 100, 110, 120, 130, 140, 150 or 160 amino acids in length.
